(19) United States
(12) Patent Application Publication
(10) Pub. No.: US 2011/0224280 A1
(43) Pub. Date: Sep. 15, 2011

(54) Pharmaceutical Composition Comprising Anti PCSK9 Oligomers

(76) Inventors: Niels Fisker Nielsen, Kgs. Lyngby (DK); Marie Wickstrom Lindholm, Malmo (SE); Ellen Marie Straarup, Birkerod (DK)

(21) Appl. No.: 12/988,289

(22) PCT Filed: Apr. 16, 2009
(86) PCT No.: PCT/EP09/54499

§ 371 Date: Jan. 7, 2011

Related U.S. Application Data
(60) Provisional application No. 61/045,579, filed on Apr. 16, 2008.
(60) Provisional application No. 61/057,417, filed on May 30, 2008.

(51) Int. Cl. A61K 31/7105; C07H 21/02; C12N 5/00; A61P 3/06

(52) U.S. Cl. 514/44 A; 536/24.5; 435/375

(57) ABSTRACT

The present invention relates to oligomer compounds (oligomers), which target PCSK9 mRNA in a cell, leading to reduced expression of PCSK9. Reduction of PCSK9 expression is beneficial for the treatment of certain medical disorders, such as HYPERCHOLESTEROLEMIA AND RELATED DISORDERS.

### RELATED CASES

The following related applications are hereby incorporated by U.S. 60/828,735, U.S. 60/972,932 and PCT/EP2007/060703.

### FIELD OF INVENTION

The present invention relates to oligomeric compounds (oligomers), that target PCSK9 mRNA in a cell, leading to reduced expression of PCSK9. Reduction of PCSK9 expression is beneficial for a range medical disorders, such as HYPERCHOLESTEROLEMIA AND RELATED DISORDERS.

### BACKGROUND

Proprotein convertase subtilisin/kexin type 9a (PCSK9) is a member of the proteinase K subfamily of subtilases. The PCSK9 gene (NARC-1) has been identified as a third locus involved in autosomal dominant hypercholesterolemia (ADH), characterised by high levels of low-density lipoprotein (LDL), xhantomas, and a high frequency of coronary heart disease. The other two loci being apolipoprotein-B (Apo-B) and the LDL receptor (LDLR). PCSK9 acts as a natural inhibitor of the LDL-receptor pathway, and both genes are regulated by depletion of cholesterol cell content and statins via sterol regulatory element-binding protein (SREBP). PCSK9 mRNA and protein levels are regulated by food intake, insulin and cell cholesterol levels (Costet et al., J. Biol. Chem. January 2006).

The human NARC1 mRNA (cDNA) sequence, which encodes human PCSK9 is shown as SEQ ID NO: 2 (NCBI Acc. No. NM_{—}174936).

The human PCSK9 polypeptide sequence (nascent) is shown as SEQ ID NO: 1 (NCBI Acc. No. NP_{—}777596). The polypeptide has a signal peptide between residues 1-30, which is co-translationally cleaved to produce a proprotein (amino acids 31-692 of SEQ ID No 2), which is subsequently cleaved by a protease to produce a mature protein corresponding to amino acids 83-692 of SEQ ID NO 2. A glycosylation site has been characterised at residue 533.

Park et al., (J. Biol. Chem. 279, pp 50630-50638, 2004) discloses that over-expression of PCSK9 reduced LDLR protein resulting in an increase in plasma LDL cholesterol, and suggests that an inhibitor of PCSK9 function may increase LDLR protein levels and enhance LDL clearance from plasma.

Rashid et al., (2005, PNAS 102, No 15, pp 5374-5379) discloses that knockout mice lacking PCSK9 manifest increased LDLR protein leading to an increased clearance of circulating lipoproteins and decreased plasma cholesterol levels, and suggests that inhibitors of PCSK9 may be useful for the treatment of hypercholesterolemia and that there may be synergy between inhibitors of PCSK9 and statins to enhance LDLRs and reduce plasma cholesterol.

WO01/57081 discloses the NARC-1 polynucleotide sequence and discloses that antisense nucleic acids can be designed using the NARC-1 polynucleotide sequence, and that such antisense nucleic acids may comprise modified nucleotides or bases, such as peptide nucleic acids.

WO2004/097047, which discloses two mutants of PCSK9 which are associated with ADH, suggests that antisense or RNAi of such PCSK9 mutants may be used for treatment of ADH.

### SUMMARY OF INVENTION

The invention provides an oligomer of between 10-30 nucleotides in length which comprises a contiguous nucleotide sequence of a total of between 10-30 nucleotides, wherein said contiguous nucleotide sequence is at least 80% (e.g., 85%, 90%, 95%, 98%, 99%) homologous to a region corresponding to the reverse complement of a mammalian PCSK9 gene or mRNA, such as SEQ ID NO: 80 or naturally occurring variant thereof. Thus, for example, the oligomer hybridizes to a single stranded nucleic acid molecule having the sequence of a portion of SEQ ID NO: 80.

The invention provides for a conjugate comprising the oligomer according to the invention, and at least one non-nucleotide or non-polynucleotide moiety covalently attached to said oligomer.

The invention provides for a pharmaceutical composition comprising the oligomer or the conjugate according to the invention, and a pharmaceutically acceptable diluent, carrier, salt or adjuvant.

The invention provides for the oligomer or the conjugate according to invention, for use as a medicament, such as for the treatment of HYPERCHOLESTEROLEMIA AND RELATED DISORDERS.

The invention provides for the use of an oligomer or the conjugate according to the invention, for the manufacture of a medicament for the treatment of HYPERCHOLESTEROLEMIA AND RELATED DISORDERS.

The invention provides for a method of treating HYPERCHOLESTEROLEMIA AND RELATED DISORDERS, said method comprising administering an oligomer, a conjugate or a pharmaceutical composition according to the invention, to a patient suffering from, or likely to suffer from HYPERCHOLESTEROLEMIA AND RELATED DISORDERS.

The term “related disorders” when referring to hypercholesterolemia refers to one or more of the conditions selected from the group consisting of: atherosclerosis, hyperlipidemia, HDL/LDL cholesterol imbalance, dyslipidemias, e.g., familial combined hyperlipidemia (FCHL), acquired hyperlipidemia, statin-resistant hypercholesterolemia, coronary artery disease (CAD), and coronary heart disease (CHD).

The invention provides for a method for the inhibition of PCSK9 in a cell which is expressing PCSK9, said method comprising administering an oligomer, or a conjugate according to the invention to said cell so as to effect the inhibition of PCSK9 in said cell.

In some aspects of the invention, the oligonucleotides of the invention have an approvable tox profile.

### FIGURE LEGENDS

FIG. 1) PCSK9 mRNA levels in Huh-7 cells measured by qPCR after transfection with anti-PCSK9 LNA containing oligonucleotides. Results shown as relative levels of PCSK9 mRNA (% of mock, i.e. transfection without oligonucleotide). Concentrations: 1, 5, or 25 nm.

FIG. 2) PCSK9 mRNA levels in Huh-7 cells measured by qPCR after transfection with anti-PCSK9 LNA containing oligonucleotides. Results shown as relative levels of PCSK9 mRNA (% of mock, i.e. transfection without oligonucleotide). Concentrations: 1, 5, or 25 nm.

FIG. 3) Specific design of compound ID 1-16. Capitals are LNA nucleotides Small letters are DNA nucleotides. 13 mers are shown as 3-8-2 (LNA-DNA-LNA), but in an equally preferred embodiment, 12 mers are 2-8-2 (LNA-DNA-LNA). In a preferred embodiment, internucleoside bonds are fully thiolated.

ₛ are Phosphothioate internucleotide bonds.
^{o} indicate oxy LNA, such as beta-D-oxy-LNA
^{m} indicate 5′ methylation (in connection with cytokines)

### DETAILED DESCRIPTION OF INVENTION

### The Oligomer

The present invention employs oligomeric compounds (referred herein as oligomers), for use in modulating the function of nucleic acid molecules encoding mammalian PCSK9, such as the PCSK9 nucleic acid shown in SEQ ID 80, and naturally occurring variants of such nucleic acid molecules encoding mammalian PCSK9. The term “oligomer” in the context of the present invention, refers to a molecule formed by covalent linkage of two or more nucleotides (i.e. an oligonucleotide). The oligomer consists or comprises of a contiguous nucleotide sequence of between 10-30 nucleotides in length.

In one embodiment, the compound of the invention does not comprise RNA (units). It is preferred that the compound according to the invention is a linear molecule or is synthesised as a linear molecule. The oligomer is a single stranded molecule, and preferably does not comprise short regions of, for example, at least 3, 4 or 5 contiguous nucleotides, which are complementary to equivalent regions within the same oligomer (i.e. duplexes)—in this regards, the oligomer is not (essentially) double stranded. In one embodiment, the oligomer is essentially not double stranded, such as is not a siRNA. In one embodiment, the oligomer of the invention may consist entirely of the contiguous nucleotide region. Thus, the oligomer is not substantially self-complementary.

### The Target

Suitably the oligomer of the invention is capable of down-regulating expression of the PCSK9 gene. In one embodiment, the oligomers of the invention bind to the target nucleic acid and effect inhibition of expression of at least 10% or 20% compared to the normal expression level, more preferably at least a 30%, 40%, 50%, 60%, 70%, 80%, 90% or 95% inhibition compared to the normal expression level. In some embodiments, such modulation is seen when using between 0.04 and 25 nM, such as between 0.8 and 20 nM concentration of the compound of the invention. In the same or a different embodiment, the inhibition of expression is less than 100%, such as less than 98% inhibition, less than 95% inhibition, less than 90% inhibition, less than 80% inhibition, such as less than 70% inhibition. Modulation of expression level may be determined by measuring protein levels, e.g. by the methods such as SDS-PAGE followed by western blotting using suitable antibodies raised against the target protein.

Alternatively, modulation of expression levels can be determined by measuring levels of mRNA, e.g. by northern blotting or quantitative RT-PCR. When measuring via mRNA levels, the level of down-regulation when using an appropriate dosage, such as between 0.04 and 25 nM, such as between 0.8 and 20 nM concentration, is, in one embodiment, typically to a level of between 10-20% the normal levels in the absence of the compound of the invention.

The invention therefore provides a method of down-regulating or inhibiting the expression of PCSK9 protein and/or mRNA in a cell which is expressing PCSK9 protein and/or mRNA, said method comprising administering the oligomer or conjugate according to the invention to said cell to down-regulating or inhibiting the expression of PCSK9 protein and/or mRNA in said cell. Suitably the cell is a mammalian cell such as a human cell. The administration may occur, in one embodiment, in vitro. The administration may occur, in one embodiment, in vivo.

The term “target nucleic acid”, as used herein refers to the DNA encoding mammalian PCSK9 polypeptide, such as human PCSK9, such as SEQ ID NO: 80. PCSK9 encoding nucleic acids or naturally occurring variants thereof, and RNA nucleic acids derived therefrom, preferably mRNA, such as pre-mRNA, although preferably mature mRNA. In one embodiment, for example when used in research or diagnostics the “target nucleic acid” may be a cDNA or a synthetic oligonucleotide derived from the above DNA or RNA nucleic acid targets. The oligomer according to the invention is preferably capable of hybridising to the target nucleic acid.

The term “naturally occurring variant thereof” refers to variants of the PCSK9 polypeptide of nucleic acid sequence which exist naturally within the defined taxonomic group, such as mammalian, such as mouse, monkey, and preferably human. Typically, when referring to “naturally occurring variants” of a polynucleotide the term also may encompass any allelic variant of the PCSK9 encoding genomic DNA which are found at the Chromosome 1; Location: 1p32.3 Mb by chromosomal translocation or duplication, and the RNA, such as mRNA derived therefrom. “Naturally occurring variants” may also include variants derived from alternative splicing of the PCSK9 mRNA. When referenced to a specific polypeptide sequence, e.g., the term also includes naturally occurring forms of the protein which may therefore be processed, e.g. by co- or post-translational modifications, such as signal peptide cleavage, proteolytic cleavage, glycosylation, etc.

### Sequences

The oligomers comprise or consist of a contiguous nucleotide sequence which corresponds to the reverse complement of a nucleotide sequence present in SEQ ID NO: 80. Thus, the oligomer can comprise or consist of a sequence selected from the group consisting of SEQ ID NOS: 1-79, SEQ ID NO's 81-84 or SEQ ID's 85-94 wherein said oligomer (or contiguous nucleotide portion thereof) may optionally have one, two, or three mismatches against said selected sequence. The oligomer may comprise or consist of a contiguous nucleotide sequence which is fully complementary (perfectly complementary) to the equivalent region of a nucleic acid which encodes a mammalian PCSK9 (e.g., SEQ ID NO:80). Thus, the oligomer can comprise or sinsist of an antisense nucleotide sequence. However, in some embodiments, the oligomer may tolerate 1, 2, 3, or 4 (or more) mismatches, when hybridising to the target sequence and still sufficiently bind to the target to show the desired effect, i.e., down-regulation of the target. Mismatches may, for example, be compensated by increased length of the oligomer nucleotide sequence and/or an increased number of nucleotide analogues, such as LNA, present within the nucleotide sequence.

In one embodiment, the contiguous nucleotide sequence comprises no more than 3, such as no more than 2 mismatches when hybridizing to the target sequence, such as to the corresponding region of a nucleic acid which encodes a mammalian PCSK9.

In one embodiment, the contiguous nucleotide sequence comprises no more than a single mismatch when hybridizing to the target sequence, such as the corresponding region of a nucleic acid which encodes a mammalian PCSK9. The nucleotide sequence of the oligomers of the invention or the contiguous nucleotide sequence is preferably at least 80% homologous to a corresponding sequence selected from the group consisting of SEQ ID NOS: 1-79, SEQ ID's 81-84 or SEQ ID's 85-94, such as at least 85%, at least 90%, at least 91%, at least 92%, at least 93%, at least 94%, at least 95%, at least 96% homologous, such as 100% homologous (identical).

The nucleotide sequence of the oligomers of the invention or the contiguous nucleotide sequence is preferably at least 80% homologous to the reverse complement of a corresponding sequence present in SEQ ID NO: 80, such as at least 85%, at least 90%, at least 91%, at least 92% at least 93%, at least 94%, at least 95%, at least 96% homologous, such as 100% homologous (identical). The nucleotide sequence of the oligomers of the invention or the contiguous nucleotide sequence is preferably at least 80% complementary to the reverse complement of a sub-sequence present in SEQ ID NO: 80, such as at least 85%, at least 90%, at least 91%, at least 92% at least 93%, at least 94%, at least 95%, at least 96% complementary, such as 100% complementary (perfectly complementary).

In one embodiment the oligomer (or contiguous nucleotide portion thereof) is selected from, or comprises, one of the sequences selected from the group consisting of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 81, 82, 83, 84, 85, 86, 87, 88, 89, 90, 91, 92, 93, 94, or a sub-sequence of at least 10 contiguous nucleotides thereof, wherein said oligomer (or contiguous nucleotide portion thereof) may optionally comprise one, two, or three mismatches when compared to the sequence.

In one embodiment the sub-sequence may consist of 11, 12, 13 or 14 contiguous nucleotides. Suitably, in one embodiment, the sub-sequence is of the same length as the contiguous nucleotide sequence of the oligomer of the invention.

However, it is recognised that, in one embodiment the nucleotide sequence of the oligomer may comprise additional 5′ or 3′ nucleotides, such as, independently, 1, 2, 3, 4 or 5 additional nucleotides 5′ and/or 3′, which are non-complementary to the target sequence. In this respect the oligomer of the invention, may, in one embodiment, comprise a contiguous nucleotide sequence which is flanked 5′ and or 3′ by additional nucleotides. In one embodiment the additional 5′ or 3′ nucleotides are naturally occurring nucleotides, such as DNA or RNA. In one embodiment, the additional 5′ or 3′ nucleotides may represent region D as referred to in the context of gapmer oligomers herein.

In one embodiment the oligomer according to the invention consists or comprises of a nucleotide sequence according to SEQ ID NO:81, or a sub-sequence of thereof.

In one embodiment the oligomer according to the invention consists or comprises of a nucleotide sequence according to SEQ ID NO:82, or a sub-sequence of thereof.

In one embodiment the oligomer according to the invention consists or comprises of a nucleotide sequence according to SEQ ID NO:83, or a sub-sequence of thereof.

In one embodiment the oligomer according to the invention consists or comprises of a nucleotide sequence according to SEQ ID NO:84, or a sub-sequence of thereof.

TABLE 4
Sequence motifs

| Sequence | SEQ ID NO |
|---|---|
| AACGCAAGGCTAGCACCAG | 81 |
| GCCTCCATTAATCAGGGAG | 82 |
| GACCATGCCTTAGA | 83 |
| GGATTGAATGCCTGGC | 84 |

When determining “homology” between the oligomers of the invention (or contiguous nucleotide sequence) and the nucleic acid which encodes the mammalian PCSK9 or the reverse complement thereof, such as those disclosed herein, the determination of homology may be made by a simple alignment with the corresponding nucleotide sequence of the compound of the invention and the corresponding region of the nucleic acid which encodes the mammalian PCSK9 (or target nucleic acid), or the reverse complement thereof, and the homology is determined by counting the number of bases which align and dividing by the total number of contiguous nucleotides in the compound of the invention, and multiplying by 100. In such a comparison, if gaps exist, it is preferable that such gaps are merely mismatches rather than areas where the number of nucleotides within the gap differ between the nucleotide sequence of the invention and the target nucleic acid.

The terms “corresponding to” and “corresponds to” refer to the comparison between the nucleotide sequence of the oligomer or contiguous nucleotide sequence and the equivalent nucleotide sequence of i) the reverse complement of the nucleic acid target, such as the mRNA which encodes the PCSK9 protein, such as SEQ ID NO: 80, and/or ii) the sequence of nucleotides provided herein such as the group consisting of SEQ ID NOS: 1-79, SEQ ID's 81-84 or SEQ ID's 85-94. Nucleotide analogues are compared directly to their equivalent or corresponding nucleotides.

The terms “corresponding nucleotide analogue” and “corresponding nucleotide” are intended to indicate that the nucleotide in the nucleotide analogue and the naturally occurring nucleotide are identical. For example, when the 2-deoxyribose unit of the nucleotide is linked to an adenine, the “corresponding nucleotide analogue” contains a pentose unit (different from 2-deoxyribose) linked to an adenine.

### Length

The oligomers comprise or consist of a contiguous nucleotide sequence of a total of between 10, 11, 12, 13, 14, 15, 16, 17, 18, 19, 20, 21, 22, 23, 24, 25, 26, 27, 28, 29 or 30 contiguous nucleotides in length.

In one embodiment, the oligomers comprise or consist of a contiguous nucleotide sequence of a total of between 10-22, such as 12-18, such as 13-17 or 12-16, such as 13, 14, 15, 16 contiguous nucleotides in length.

In one embodiment, the oligomers comprise or consist of a contiguous nucleotide sequence of a total of 10, 11, 12, 13, or 14 contiguous nucleotides in length.

In one embodiment, the oligomer according to the invention consists of no more than 22 nucleotides, such as no more than 20 nucleotides, such as no more than 18 nucleotides, such as 15, 16 or 17 nucleotides. In one embodiment the oligomer of the invention comprises less than 20 nucleotides.

### Nucleotide Analogues

The term “nucleotide” as used herein, refers to a glycoside comprising a sugar moiety, a base moiety and a covalently linked phosphate group and covers both naturally occurring nucleotides, such as DNA or RNA, preferably DNA, and non-naturally occurring nucleotides comprising modified sugar and/or base moieties, which are also referred to as “nucleotide analogues” herein.

Non-naturally occurring nucleotides include nucleotides which have modified sugar moieties, such as bicyclic nucleotides or 2′ modified nucleotides, such as 2′ substituted nucleotides.

“Nucleotide analogues” are variants of natural nucleotides, such as DNA or RNA nucleotides, by virtue of modifications in the sugar and/or base moieties. Analogues could in principle be merely “silent” or “equivalent” to the natural nucleotides in the context of the oligonucleotide, i.e. have no functional effect on the way the oligonucleotide works to inhibit target gene expression. Such “equivalent” analogues may nevertheless be useful if, for example, they are easier or cheaper to manufacture, or are more stable to storage or manufacturing conditions, or represent a tag or label. Preferably, however, the analogues will have a functional effect on the way in which the oligomer works to inhibit expression; for example by producing increased binding affinity to the target and/or increased resistance to intracellular nucleases and/or increased ease of transport into the cell. Specific examples of nucleoside analogues are described by e.g. Freier & Altmann; Nucl. Acid Res., 1997, 25, 4429-4443 and Uhlmann; Curr. Opinion in Drug Development, 2000, 3(2), 293-213, and in Scheme 1:

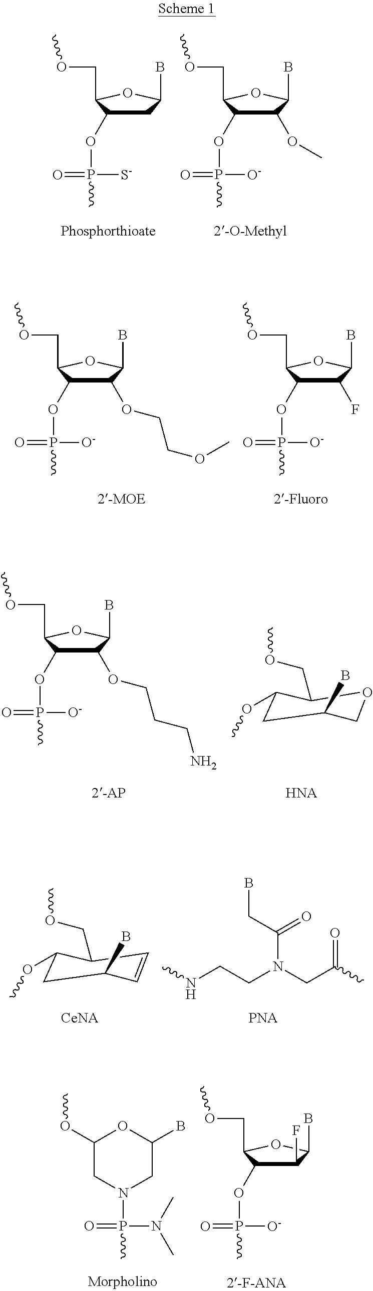

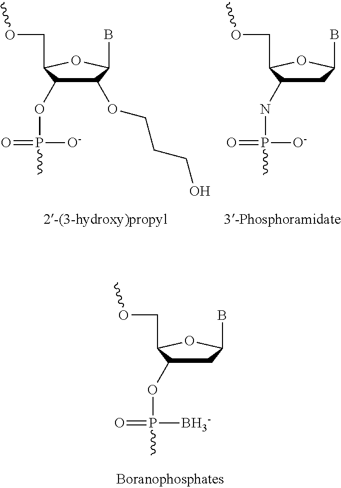

The oligomer may thus comprise or consist of a simple sequence of natural occurring nucleotides—preferably 2′-deoxynucleotides (referred to here generally as “DNA”), but also possibly ribonucleotides (referred to here generally as “RNA”), or a combination of such naturally occurring nucleotides and one or more non-naturally occurring nucleotides, i.e. nucleotide analogues. Such nucleotide analogues may suitably enhance the affinity of the oligomer for the target sequence.

Examples of suitable and preferred nucleotide analogues are provided by PCT/DK2006/000512 or are referenced therein.

Incorporation of affinity-enhancing nucleotide analogues in the oligomer, such as LNA or 2′-substituted sugars, can allow the size of the specifically binding oligomer to be reduced, and may also reduce the upper limit to the size of the oligomer before non-specific or aberrant binding takes place.

In one embodiment the oligomer comprises at least 2 nucleotide analogues. In some embodiments, the oligomer comprises from 3-8 nucleotide analogues, e.g. 6 or 7 nucleotide analogues. In the by far most preferred embodiments, at least one of said nucleotide analogues is a locked nucleic acid (LNA); for example at least 3 or at least 4, or at least 5, or at least 6, or at least 7, or 8, of the nucleotide analogues may be LNA. In some embodiments all the nucleotides analogues may be LNA.

It will be recognised that when referring to a preferred nucleotide sequence motif or nucleotide sequence, which consists of only nucleotides, the oligomers of the invention which are defined by that sequence may comprise a corresponding nucleotide analogue in place of one or more of the nucleotides present in said sequence, such as LNA units or other nucleotide analogues, which raise the duplex stability/Tₘ of the oligomer/target duplex (i.e. affinity enhancing nucleotide analogues).

In one embodiment, any mismatches between the nucleotide sequence of the oligomer and the target sequence are preferably found in regions outside the affinity enhancing nucleotide analogues, such as region B as referred to herein, and/or region D as referred to herein, and/or at the site of non modified such as DNA nucleotides in the oligonucleotide, and/or in regions which are 5′ or 3′ to the contiguous nucleotide sequence.

Examples of such modification of the nucleotide include modifying the sugar moiety to provide a 2′-substituent group or to produce a bridged (locked nucleic acid) structure which enhances binding affinity and may also provide increased nuclease resistance.

A preferred nucleotide analogue is LNA, such as oxy-LNA (such as beta-D-oxy-LNA, and alpha-L-oxy-LNA), and/or amino-LNA (such as beta-D-amino-LNA and alpha-L-amino-LNA) and/or thio-LNA (such as beta-D-thio-LNA and alpha-L-thio-LNA) and/or ENA (such as beta-D-ENA and alpha-L-ENA).

In some embodiments the nucleotide analogues present within the oligomer of the invention (such as in regions A and C mentioned herein) are independently selected from, for example: 2′-O-alkyl-RNA units, 2′-amino-DNA units, 2′-fluoro-DNA units, LNA units, arabino nucleic acid (ANA) units, 2′-fluoro-ANA units, HNA units, INA (intercalating nucleic acid—Christensen, 2002. Nucl. Acids. Res. 2002 30: 4918-4925, hereby incorporated by reference) units and 2′MOE units. In one embodiment there is only one of the above types of nucleotide analogues present in the oligomer of the invention, or contiguous nucleotide sequence thereof.

In one embodiment the nucleotide analogues are 2′-O-methoxyethyl-RNA (2′MOE), 2′-fluoro-DNA monomers or LNA nucleotide analogues, and as such the oligonucleotide of the invention may comprise nucleotide analogues which are independently selected from these three types of analogue, or may comprise only one type of analogue selected from the three types. In one embodiment at least one of said nucleotide analogues is 2′-MOE-RNA, such as 2, 3, 4, 5, 6, 7, 8, 9 or 10 2′-MOE-RNA nucleotide units. In one embodiment at least one of said nucleotide analogues is 2′-fluoro DNA, such as 2, 3, 4, 5, 6, 7, 8, 9 or 10 2′-fluoro-DNA nucleotide units.

In one embodiment, the oligomer according to the invention comprises at least one Locked Nucleic Acid (LNA) unit, such as 1, 2, 3, 4, 5, 6, 7, or 8 LNA units, such as between 3-7 or 4 to 8 LNA units, or 3, 4, 5, 6 or 7 LNA units. In one embodiment, all the nucleotide analogues are LNA. In some embodiments, the oligomer may comprise both beta-D-oxy-LNA, and one or more of the following LNA units: thio-LNA, amino-LNA, oxy-LNA, and/or ENA in either the beta-D or alpha-L configurations or combinations thereof. In one embodiment all LNA cytosine units are 5′ methyl-Cytosine. In one embodiment of the invention, the oligomer may comprise both LNA and DNA units. Preferably the combined total of LNA and DNA units is 10-25, preferably 10-20, even more preferably 12-16.

In one embodiment of the invention, the nucleotide sequence of the oligomer, such as the contiguous nucleotide sequence consists of at least one LNA and the remaining nucleotide units are DNA units. In one embodiment the oligomer comprises only LNA nucleotide analogues and naturally occurring nucleotides (such as RNA or DNA, most preferably DNA nucleotides), optionally with modified internucleotide linkages such as phosphorothioate.

The term “nucleobase” refers to the base moiety of a nucleotide and covers both naturally occurring a well as non-naturally occurring variants. Thus, “nucleobase” covers not only the known purine and pyrimidine heterocycles but also heterocyclic analogues and tautomeres thereof.

Examples of nucleobases include, but are not limited to adenine, guanine, cytosine, thymidine, uracil, xanthine, hypoxanthine, 5-methylcytosine, isocytosine, pseudoisocytosine, 5-bromouracil, 5-propynyluracil, 6-aminopurine, 2-aminopurine, inosine, diaminopurine, and 2-chloro-6-aminopurine.

In one embodiment, at least one of the nucleobases present in the oligomer is a modified nucleobase selected from the group consisting of 5-methylcytosine, isocytosine, pseudoisocytosine, 5-bromouracil, 5-propynyluracil, 6-aminopurine, 2-aminopurine, inosine, diaminopurine, and 2-chloro-6-aminopurine.

### LNA

The term “LNA” refers to a bicyclic nucleotide analogue, known as “Locked Nucleic Acid”. It may refer to an LNA monomer, or, when used in the context of an “LNA oligonucleotide” refers to an oligonucleotide containing one or more such bicyclic nucleotide analogues.

The LNA used in the oligonucleotide compounds of the invention preferably has the structure of the general formula I

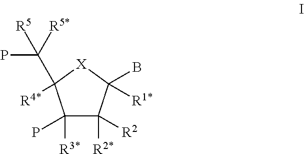

wherein X is selected from —O—, —S—, —N(R^{N*})—, —C(R⁶R^{6*})—;
B is selected from hydrogen, optionally substituted C₁₋₄-alkoxy, optionally substituted C₁₋₄-alkyl, optionally substituted C₁₋₄-acyloxy, nucleobases, DNA intercalators, photochemically active groups, thermochemically active groups, chelating groups, reporter groups, and ligands;
P designates the radical position for an internucleotide linkage to a succeeding monomer, or a 5′-terminal group, such internucleotide linkage or 5′-terminal group optionally including the substituent R⁵ or equally applicable the substituent R^{5*};
P* designates an internucleotide linkage to a preceding monomer, or a 3′-terminal group;
R^{4*} and R^{2*} together designate a biradical consisting of 1-4 groups/atoms selected from —C(R^{a}R^{b})—, —C(R^{a})═C(R^{b})—, —C(R^{a})═N—, —O—, —Si(R^{a})₂—, —S—, —SO₂—, —N(R^{a})—, and >C═Z,
wherein Z is selected from —O—, —S—, and —N(R^{a})—,
and R^{a} and R^{b} each is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkoxyalkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted and where two geminal substituents R^{a} and R^{b} together may designate optionally substituted methylene (═CH₂), and
each of the substituents R^{1*}, R², R³, R⁵, R^{5*}, R⁶ and R^{6*}, which are present is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkoxyalkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted, and where two geminal substituents together may designate oxo, thioxo, imino, or optionally substituted methylene, or together may form a spiro biradical consisting of a 1-5 carbon atom(s) alkylene chain which is optionally interrupted and/or terminated by one or more heteroatoms/groups selected from —O—, —S—, and —(NR^{N})—where R^{N} is selected from hydrogen and C₁₋₄-alkyl, and where two adjacent (non-geminal) substituents may designate an additional bond resulting in a double bond; and
R^{N*}, when present and not involved in a biradical, is selected from hydrogen and C₁₋₄-alkyl; and basic salts and acid addition salts thereof;

In one embodiment R^{5*} is selected from H, —CH₃, —CH₂—CH₃, —CH₂—O—CH₃, and —CH═CH₂.

In one embodiment, R^{4*} and R^{2*} together designate a biradical selected from —C(R^{a}R^{b})—O—, —C(R^{a}R^{b})—C(R^{c}R^{d})—O—, —C(R^{a}R^{b})—C(R^{c}R^{d})—C(R^{e}R^{f})—O—, —C(R^{a}R^{b})—O—C(R^{c}R^{d})—, —C(R^{a}R^{b})—O—C(R^{c}R^{d})—O—, —C(R^{a}R^{b})—C(R^{c}R^{d})—, —C(R^{a}R^{b})—C(R^{c}R^{d})—C(R^{e}R^{f})—, —C(R^{a})═C(R^{b})—C(R^{c}R^{d})—, —C(R^{a}R^{b})—N(R^{c})—, —C(R^{a}R^{b})—C(R^{c}R^{d})—N(R^{e})—, —C(R^{a}R^{b})—N(R^{c})—O—, and —C(R^{a}R^{b})—S—, —C(R^{a}R^{b})—C(R^{c}R^{d})—S—,

wherein R^{a}, R^{b}, R^{c}, R^{d}, R^{e}, and R^{f} each is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkoxyalkyl, C₂₋₁₂-alkenyloxy, carboxy, C₁₋₁₂-alkoxycarbonyl, C₁₋₁₂-alkylcarbonyl, formyl, aryl, aryl-oxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted and where two geminal substituents R^{a} and R^{b} together may designate optionally substituted methylene (═CH₂),

In a further embodiment R^{4*} and R^{2*} together designate a biradical (bivalent group) selected from —CH₂—O—,

—CH₂—S—, —CH₂—NH—, —CH₂—N(CH₃)—, —CH₂—CH₂—O—, —CH₂—CH(CH₃)—, —CH₂—CH₂—S—, —CH₂—CH₂—NH—, —CH₂—CH₂—CH₂—, —CH₂—CH₂—CH₂—O—, —CH₂—CH₂—CH(CH₃)—, —CH═CH—CH₂—, —CH₂—O—CH₂—O—, —CH₂—NH—O—, —CH₂—N(CH₃)—O—, —CH₂—O—CH₂—, —CH(CH₃)—O—, —CH(CH₂—O—CH₃)—O—.

For all chiral centers, asymmetric groups may be found in either R or S orientation.

Preferably, the LNA used in the oligomer of the invention comprises at least one

LNA unit according to any of the formulas

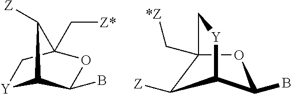

wherein Y is —O—, —O—CH₂—, —S—, —NH—, or N(R^{H}); Z and Z* are independently selected among an internucleotide linkage, a terminal group or a protecting group; B constitutes a natural or non-natural nucleotide base moiety, and R^{H} is selected from hydrogen and C₁₋₄-alkyl.

Specifically preferred LNA units are shown in scheme 2:

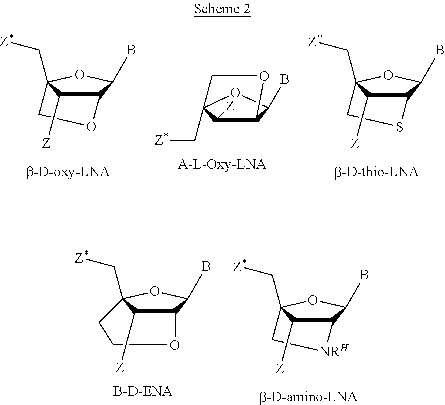

The term “thio-LNA” comprises a locked nucleotide in which at least one of X or Y in the general formula above is selected from S or —CH₂—S—. Thio-LNA can be in both beta-D and alpha-L-configuration.

The term “amino-LNA” comprises a locked nucleotide in which at least one of X or Y in the general formula above is selected from —N(H)—, N(R)—, CH₂—N(H)—, and —CH₂—N(R)— where R is selected from hydrogen and C₁₋₄-alkyl. Amino-LNA can be in both beta-D and alpha-L-configuration.

The term “oxy-LNA” comprises a locked nucleotide in which at least one of X or Y in the general formula above represents —O— or —CH₂—O—. Oxy-LNA can be in both beta-D and alpha-L-configuration.

The term “ENA” comprises a locked nucleotide in which Y in the general formula above is —CH₂—O— (where the oxygen atom of —CH₂—O— is attached to the 2′-position relative to the base B).

In a preferred embodiment LNA is selected from beta-D-oxy-LNA, alpha-L-oxy-LNA, beta-D-amino-LNA and beta-D-thio-LNA, in particular beta-D-oxy-LNA.

### RNAse H Recruitment

It is recognised that an oligomeric compound may function via non RNase mediated degradation of target mRNA, such as by steric hindrance of translation, or other methods, however, the preferred oligomers of the invention are capable of recruiting an (endo)ribonuclease (RNase), such as RNase H.

It is preferable that the oligomer, or contiguous nucleotide sequence, comprises of a region of at least 6, such as at least 7 consecutive nucleotide units, such as at least 8 or at least 9 consecutive nucleotide units (residues), including 7, 8, 9, 10, 11, 12, 13, 14, 15 or 16 consecutive nucleotides, which, when formed in a duplex with the complementary target RNA is capable of recruiting RNase. The contiguous sequence which is capable of recruiting RNAse may be region B as referred to in the context of a gapmer as described herein. In one embodiment the size of the contiguous sequence which is capable of recruiting RNAse, such as region B, may be higher, such as 10, 11, 12, 13, 14, 15, 16, 17, 18, 19 or 20 nucleotide units.

EP 1 222 309 provides in vitro methods for determining RNaseH activity, which may be used to determine the ability to recruit RNaseH. A oligomer is deemed capable of recruiting RNase H if, when provided with the complementary RNA target, it has an initial rate, as measured in pmol/l/min, of at least 1%, such as at least 5%, such as at least 10% or less than 20% of the equivalent DNA only oligonucleotide, with no 2′ substitutions, with phosphorothioate linkage groups between all nucleotides in the oligonucleotide, using the methodology provided by Example 91-95 of EP 1 222 309.

In one embodiment, an oligomer is deemed essentially incapable of recruiting RNaseH if, when provided with the complementary RNA target, and RNaseH, the RNaseH initial rate, as measured in pmol/l/min, is less than 1%, such as less than 5%, such as less than 10% or less than 20% of the initial rate determined using the equivalent DNA only oligonucleotide, with no 2′ substitutions, with phosphorothioate linkage groups between all nucleotides in the oligonucleotide, using the methodology provided by Example 91-95 of EP 1 222 309.

In other embodiments, an oligomer is deemed capable of recruiting RNaseH if, when provided with the complementary RNA target, and RNaseH, the RNaseH initial rate, as measured in pmol/l/min, is at least 20%, such as at least 40%, such as at least 60%, such as at least 80% of the initial rate determined using the equivalent DNA only oligonucleotide, with no 2′ substitutions, with phosphorothioate linkage groups between all nucleotides in the oligonucleotide, using the methodology provided by Example 91-95 of EP 1 222 309.

Typically the region of the oligomer which forms the consecutive nucleotide units which, when formed in a duplex with the complementary target RNA is capable of recruiting RNase consists of nucleotide units which form a DNA/RNA like duplex with the RNA target—and include both DNA units and LNA units which are in the alpha-L configuration, particularly preferred being alpha-L-oxy LNA.

The oligomer of the invention may comprise a nucleotide sequence which comprises both nucleotides and nucleotide analogues, and may be in the form of a gapmer, a headmer or a mixmer.

A headmer is defined by a contiguous stretch of non-RNase recruiting nucleotide analogues at the 5′-end followed by a contiguous stretch of DNA or modified nucleotide units recognizable and cleavable by the RNase towards the 3′-end (such as at least 7 such nucleotides), and a tailmer is defined by a contiguous stretch of DNA or modified nucleotides recognizable and cleavable by the RNase at the 5′-end (such as at least 7 such nucleotides), followed by a contiguous stretch of non-RNase recruiting nucleotide analogues towards the 3′-end. Other chimeras according to the invention, called mixmers consisting of an alternate composition of DNA or modified nucleotides recognizable and cleavable by RNase and non-RNase recruiting nucleotide analogues. Some nucleotide analogues may also be able to mediate RNaseH binding and cleavage. Since α-L-LNA recruits RNaseH activity to a certain extent, smaller gaps of DNA or modified nucleotides recognizable and cleavable by the RNaseH for the gapmer construct might be required, and more flexibility in the mixmer construction might be introduced.

### Gapmer Design

Preferably, the oligomer of the invention is a gapmer. A gapmer oligomer is an oligomer which comprises a contiguous stretch of nucleotides which is capable of recruiting an RNAse, such as RNAseH, such as a region of at least 6 or 7 DNA nucleotides, referred to herein in as region B, wherein region B is flanked both 5′ and 3′ by regions of affinity enhancing nucleotide analogues, such as between 1-6 nucleotide analogues 5′ and 3′ to the contiguous stretch of nucleotides which is capable of recruiting RNAse—these regions are referred to as regions A and C respectively.

Preferably the gapmer comprises a (poly)nucleotide sequence of formula (5′ to 3′), A-B-C, or optionally A-B-C-D or D-A-B-C, wherein; region A (5′ region) consists or comprises of at least one nucleotide analogue, such as at least one LNA unit, such as between 1-6 nucleotide analogues, such as LNA units, and; region B consists or comprises of at least five consecutive nucleotides which are capable of recruiting RNAse (when formed in a duplex with a complementary RNA molecule, such as the mRNA target), such as DNA nucleotides, and; region C (3′ region) consists or comprises of at least one nucleotide analogue, such as at least one LNA unit, such as between 1-6 nucleotide analogues, such as LNA units, and; region D, when present consists or comprises of 1, 2 or 3 nucleotide units, such as DNA nucleotides.

In one embodiment, region A consists of 1, 2, 3, 4, 5 or 6 nucleotide analogues, such as LNA units, such as between 2-5 nucleotide analogues, such as 2-5 LNA units, such as 3 or 4 nucleotide analogues, such as 3 or 4 LNA units; and/or region C consists of 1, 2, 3, 4, 5 or 6 nucleotide analogues, such as LNA units, such as between 2-5 nucleotide analogues, such as 2-5 LNA units, such as 3 or 4 nucleotide analogues, such as 3 or 4 LNA units.

In one embodiment B consists or comprises of 5, 6, 7, 8, 9, 10, 11 or 12 consecutive nucleotides which are capable of recruiting RNAse, or between 6-10, or between 7-9, such as 8 consecutive nucleotides which are capable of recruiting RNAse. In one embodiment region B consists or comprises at least one DNA nucleotide unit, such as 1-12 DNA units, preferably between 4-12 DNA units, more preferably between 6-10 DNA units, such as between 7-10 DNA units, most preferably 8, 9 or 10 DNA units.

In one embodiment region A consist of 3 or 4 nucleotide analogues, such as LNA, region B consists of 7, 8, 9 or 10 DNA units, and region C consists of 3 or 4 nucleotide analogues, such as LNA. Such designs include (A-B-C) 3-10-3, 3-10-4, 4-10-3, 3-9-3, 3-9-4, 4-9-3, 3-8-3, 3-8-4, 4-8-3, 3-7-3, 3-7-4, 4-7-3, and may further include region D, which may have one or 2 nucleotide units, such as DNA units.

Further gapmer designs are disclosed in WO2004/046160 and are hereby incorporated by reference.

US provisional application, 60/977,409, hereby incorporated by reference, refers to ‘shortmer’ gapmer oligomers, which, in one embodiment may be the gapmer oligomer according to the present invention.

In one embodiment the oligomer is consisting of a contiguous nucleotide sequence of a total of 10, 11, 12, 13 or 14 nucleotide units, wherein the contiguous nucleotide sequence is of formula (5′-3′), A-B-C, or optionally A-B-C-D or D-A-B-C, wherein; A consists of 1, 2 or 3 nucleotide analogue units, such as LNA units; B consists of 7, 8 or 9 contiguous nucleotide units which are capable of recruiting RNAse when formed in a duplex with a complementary RNA molecule (such as a mRNA target); and C consists of 1, 2 or 3 nucleotide analogue units, such as LNA units. When present, D consists of a single DNA unit.

In one embodiment A consists of 1 LNA unit. In one embodiment A consists of 2 LNA units. In one embodiment A consists of 3 LNA units. In one embodiment C consists of 1 LNA unit. In one embodiment C consists of 2 LNA units. In one embodiment C consists of 3 LNA units. In one embodiment B consists of 7 nucleotide units. In one embodiment B consists of 8 nucleotide units. In one embodiment B consists of 9 nucleotide units. In one embodiment B comprises of between 1-9 DNA units, such as 2, 3, 4, 5, 6, 7 or 8 DNA units. In one embodiment B consists of DNA units. In one embodiment B comprises of at least one LNA unit which is in the alpha-L configuration, such as 2, 3, 4, 5, 6, 7, 8 or 9 LNA units in the alpha-L-configuration. In one embodiment B comprises of at least one alpha-L-oxy LNA unit or wherein all the LNA units in the alpha-L-configuration are alpha-L-oxy LNA units. In one embodiment the number of nucleotides present in A-B-C are selected from the group consisting of (nucleotide analogue units—region B—nucleotide analogue units): 1-8-1, 1-8-2, 2-8-1, 2-8-2, 3-8-3, 2-8-3, 3-8-2, 4-8-1, 4-8-2, 1-8-4, 2-8-4, or; 1-9-1, 1-9-2, 2-9-1, 2-9-2, 2-9-3, 3-9-2, 1-9-3, 3-9-1, 4-9-1, 1-9-4, or; 1-10-1, 1-10-2, 2-10-1, 2-10-2, 1-10-3, 3-10-1. In one embodiment the number of nucleotides in A-B-C are selected from the group consisting of: 2-7-1, 1-7-2, 2-7-2, 3-7-3, 2-7-3, 3-7-2, 3-7-4, and 4-7-3. In one embodiment both A and C consists of two LNA units each, and B consists of 8 or 9 nucleotide units, preferably DNA units.

### Internucleotide Linkages

The terms “linkage group” or “internucleotide linkage” are intended to mean a group capable of covalently coupling together two nucleotides, two nucleotide analogues, and a nucleotide and a nucleotide analogue, etc. Specific and preferred examples include phosphate groups and phosphorothioate groups. The nucleotides of the oligomer of the invention or contiguous nucleotides sequence thereof are coupled together via linkage groups. Suitably each nucleotide is linked to the 3′ adjacent nucleotide via a linkage group.

Suitable internucleotide linkages include those listed within PCT/DK2006/000512, for example the internucleotide linkages listed on the first paragraph of page 34 of PCT/DK2006/000512 (hereby incorporated by reference).

It is, in one embodiment, preferred to modify the internucleotide linkage from its normal phosphodiester to one that is more resistant to nuclease attack, such as phosphorothioate or boranophosphate—these two, being cleavable by RNase H, also allow that route of antisense inhibition in reducing the expression of the target gene.

Suitable sulphur (S) containing internucleotide linkages as provided herein may be preferred. Phosphorothioate internucleotide linkages are also preferred, particularly for the gap region (B) of gapmers. Phosphorothioate linkages may also be used for the flanking regions (A and C, and for linking A or C to D, and within region D, as appropriate).

Regions A, B and C, may however comprise internucleotide linkages other than phosphorothioate, such as phosphodiester linkages, particularly, for instance when the use of nucleotide analogues protects the internucleotide linkages within regions A and C from endo-nuclease degradation—such as when regions A and C comprise LNA nucleotides.

The internucleotide linkages in the oligomer may be phosphodiester, phosphorothioate or boranophosphate so as to allow RNase H cleavage of targeted RNA. Phosphorothioate is preferred, for improved nuclease resistance and other reasons, such as ease of manufacture.

In one aspect of the oligomer of the invention, the nucleotides and/or nucleotide analogues are linked to each other by means of phosphorothioate groups.

In some embodiments region A comprises at least one phosphodiester linkage between two nucleotide analogue units, or a nucleotide analogue unit and a nucleotide unit of Region B. In some embodiments region C comprises at least one phosphodiester linkage between two nucleotide analogue units, or a nucleotide analogue unit and a nucleotide unit of Region B.

In some embodiments, region C comprises at least one phosphodiester linkage between a nucleotide analogue unit of region C and a nucleotide unit of Region D, i.e. the linkage group between regions C and D is a phosphodiester.

In some embodiments the internucleotide linkage between the 3′ nucleotide analogue of region A and the 5′ nucleotide of region B is a phosphodiester.

In some embodiments the internucleotide linkage between the 3′ nucleotide of region B and the 5′ nucleotide analogue of region C is a phosphodiester.

In some embodiments the internucleotide linkage between the two adjacent nucleotide analogues at the 5′ end of region A are phosphodiester.

In some embodiments the internucleotide linkage between the two adjacent nucleotide analogues at the 3′ end of region C is phosphodiester.

In some embodiments the internucleotide linkage between the two adjacent nucleotide analogues at the 3′ end of region A is phosphodiester.

In some embodiments the internucleotide linkage between the two adjacent nucleotide analogues at the 5′ end of region C is phosphodiester.

In some embodiments region A has a length of 4 nucleotide analogues and the internucleotide linkage between the two middle nucleotide analogues of region A is phosphodiester.

In some embodiments region C has a length of 4 nucleotide analogues and internucleotide linkage between the two middle nucleotide analogues of region C is phosphodiester.

In some embodiments all the internucleotide linkages between nucleotide analogues present in the compound of the invention are phosphodiester.

In some embodiments, such as the embodiments referred to above, where suitable and not specifically indicated, all remaining internucleotide linkages are either phosphodiester or phosphorothioate, or a mixture thereof.

In some embodiments all the internucleotide linkage groups are phosphorothioate.

When referring to specific gapmer oligonucleotide sequences, such as those provided herein it will be understood that, in one embodiment, when the linkages are phosphorothioate linkages, alternative linkages, such as those disclosed herein may be used, for example phosphate (phosphodiester) linkages may be used, particularly for linkages between nucleotide analogues, such as LNA, units. Likewise, when referring to specific gapmer oligonucleotide sequences, such as those provided herein, when the C residues are annotated as 5′ methyl modified cytosine, in one embodiment, one or more of the Cs present in the oligonucleotide may be unmodified C residues.

### Oligomeric Compounds

The sequences of the oligomers of the invention may be selected from the group consisting of: SEQ IDS. 1-79, SEQ ID's 81-84 or SEQ ID's 85-94. Some preferred oligomer designs are presented in Table 2,

### Conjugates

In one embodiment, the oligomer of the invention may comprise both a polynucleotide region, i.e. a nucleotide region, which typically consists of a contiguous sequence of nucleotides, and a further non-nucleotide region. When referring to the oligomer of the invention consisting of a contiguous nucleotide sequence, the compound may comprise non-nucleotide components, such as a conjugate component.

In one embodiment of the invention the oligomeric compound is linked to ligands/conjugates, which may be used, e.g. to increase the cellular uptake of oligomeric compounds. PCT/DK2006/000512 provides suitable ligands and conjugates, which are hereby incorporated by reference.

The invention also provides for a conjugate comprising the compound according to the invention as herein described, and at least one non-nucleotide or non-polynucleotide moiety covalently attached to said compound. Therefore, in one embodiment where the compound of the invention consists of a specified nucleic acid, as herein disclosed, the compound may also comprise at least one non-nucleotide or non-polynucleotide moiety (e.g. not comprising one or more nucleotides or nucleotide analogues) covalently attached to said compound.

Conjugates may enhance the activity, cellular distribution or cellular uptake of the oligomer of the invention. Such moieties include, but are not limited to, antibodies, polypeptides, lipid moieties such as a cholesterol moiety, cholic acid, a thioether, e.g. Hexyl-s-tritylthiol, a thiocholesterol, an aliphatic chain, e.g., dodecandiol or undecyl residues, a phospholipids, e.g., di-hexadecyl-rac-glycerol or triethylammonium 1,2-di-o-hexadecyl-rac-glycero-3-h-phosphonate, a polyamine or a polyethylene glycol chain, an adamantane acetic acid, a palmityl moiety, an octadecylamine or hexylamino-carbonyl-oxycholesterol moiety.

The oligomers of the invention may also be conjugated to active drug substances, for example, aspirin, ibuprofen, a sulfa drug, an antidiabetic, an antibacterial or an antibiotic.

In one embodiment the conjugate is a sterol, such as cholesterol.

### Compositions

The oligomer of the invention may be used in pharmaceutical formulations and compositions. Suitably, such compositions comprise a pharmaceutically acceptable diluent, carrier, salt or adjuvant. PCT/DK2006/000512 provides suitable and preferred pharmaceutically acceptable diluent, carrier and adjuvants—which are hereby incorporated by reference. Suitable dosages, formulations, administration routes, compositions, dosage forms, combinations with other therapeutic agents, pro-drug formulations are also provided in PCT/DK2006/000512-which are also hereby incorporated by reference.

### Applications

The oligomers of the invention may be utilized as research reagents for, for example, diagnostics, therapeutics and prophylaxis.

In research, such oligomers may be used to specifically inhibit the synthesis of PCSK9 protein (typically by degrading or inhibiting the mRNA and thereby prevent protein formation) in cells and experimental animals thereby facilitating functional analysis of the target or an appraisal of its usefulness as a target for therapeutic intervention.

In diagnostics the oligomers may be used to detect and quantitate PCSK9 expression in cell and tissues by northern blotting, in-situ hybridisation or similar techniques.

For therapeutics, an animal or a human, suspected of having a disease or disorder, which can be treated by modulating the expression of PCSK9 is treated by administering oligomeric compounds in accordance with this invention.

Further provided are methods of treating a mammal, such as treating a human, suspected of having or being prone to a disease or condition, associated with expression of PCSK9 by administering a therapeutically or prophylactically effective amount of one or more of the oligomers or compositions of the invention.

The invention also provides for the use of the compound or conjugate of the invention as described for the manufacture of a medicament for the treatment of a disorder as referred to herein, or for a method of the treatment of as a disorder as referred to herein.

The invention also provides for a method for treating a disorder as referred to herein said method comprising administering a compound according to the invention as herein described, and/or a conjugate according to the invention, and/or a pharmaceutical composition according to the invention to a patient in need thereof.

### Medical Indications

The oligomers and other compositions according to the invention can be used for the treatment of conditions associated with over expression or expression of mutated version of the PCSK9.

The invention further provides use of a compound of the invention in the manufacture of a medicament for the treatment of a disease, disorder or condition as referred to herein.

Generally stated, one aspect of the invention is directed to a method of treating a mammal suffering from or susceptible to conditions associated with abnormal levels of PCSK9, comprising administering to the mammal and therapeutically effective amount of an oligomer targeted to PCSK9 that comprises one or more LNA units.

The disease or disorder, as referred to herein, may, in one embodiment be associated with a mutation in the PCSK9 gene or a gene whose protein product is associated with or interacts with PCSK9. Therefore, in one embodiment, the target mRNA is a mutated form of the PCSK9 sequence.

An interesting aspect of the invention is directed to the use of an oligomer (compound) as defined herein or a conjugate as defined herein for the preparation of a medicament for the treatment of a disease, disorder or condition as referred to herein.

The methods of the invention are preferably employed for treatment or prophylaxis against diseases caused by abnormal levels of PCSK9.

Alternatively stated, in one embodiment, the invention is furthermore directed to a method for treating abnormal levels of PCSK9, said method comprising administering a oligomer of the invention, or a conjugate of the invention or a pharmaceutical composition of the invention to a patient in need thereof.

The invention also relates to an oligomer, a composition or a conjugate as defined herein for use as a medicament.

The invention further relates to use of a compound, composition, or a conjugate as defined herein for the manufacture of a medicament for the treatment of abnormal levels of PCSK9 or expression of mutant forms of PCSK9 (such as allelic variants, such as those associated with one of the diseases referred to herein).

Moreover, the invention relates to a method of treating a subject suffering from a disease or condition such as those referred to herein.

A patient who is in need of treatment is a patient suffering from or likely to suffer from the disease or disorder.

In one embodiment, the term ‘treatment’ as used herein refers to both treatment of an existing disease (e.g. a disease or disorder as herein referred to), or prevention of a disease, i.e. prophylaxis. It will therefore be recognised that treatment as referred to herein may, in one embodiment, be prophylactic.

### Embodiments

The following embodiments of the present invention may be used in combination with the other embodiments described herein.

Embodiments of the Invention: The following list refers to some, non-limiting, aspects of the invention which may be combined with the other embodiments referred to in the specification and claims:

1. A compound consisting of a contiguous sequence of a total of between 10-50 nucleobases, wherein said contiguous nucleobase sequence is at least 80% homologous to the reverse complement of a corresponding region of a nucleic acid which encodes a mammalian PCSK9.
2. A compound according to embodiment 1, wherein said compound consists of a contiguous sequence of a total of between 10-30 nucleobases, wherein said compound comprises a subsequence of at least 8 contiguous nucleobases, wherein said subsequence corresponds to the reverse complement of a contiguous sequence which is present in the nucleic acids which encode mammalian PCSK9, wherein said subsequence may comprise no more than one mismatch when compared to the reverse complement of a corresponding sequence present in the nucleic acid which encodes said mammalian PCSK9.
3. The compound according to embodiment 1 or 2, wherein said nucleic acid which encodes said mammalian PCSK9, is naturally present in a mammal selected form the group consisting of: a rodent, a mouse, a rat, a primate, a human, a monkey and a chimpanzee.
4. The compound according to embodiment 1 or 2, wherein said nucleic acid which encodes said mammalian PCSK9, is naturally present in a human being.
5. The compound according to any one of embodiments 2-4, wherein said compound comprises a 5′ and/or a 3′ flanking nucleobase sequence, which is/are contiguous to said subsequence, wherein said flanking sequence or sequences consist of a total of between 2 and 22 nucleobase units, which when combined with said sub-sequence, the combined contiguous nucleobase sequence is at least 80% homologous, such as at least 85% homologous, such as at least 90% homologous, such as at least 95% homologous, such as at least 97% homologous, such as 100% homologous to the reverse complement of a corresponding sequence of said nucleic acid which encodes said mammalian PCSK9.
6. The compound according to any one of embodiments 2 to 5, wherein said subsequence or combined nucleobase sequence comprises a contiguous sequence of at least 7 nucleobase residues which, when formed in a duplex with the complementary target RNA corresponding to said nucleic acid which encodes said mammalian PCSK9, are capable of recruiting RNaseH.
7. The compound according to embodiment 6, wherein said subsequence or combined nucleobase sequence comprises of a contiguous sequence of at least 8, at least 9 or at least 10 nucleobase residues which, when formed in a duplex with the complementary target RNA corresponding to said nucleic acid which encodes said mammalian PCSK9, are capable of recruiting RNaseH.
8. The compound according to any one of the preceding embodiments wherein said subsequence is at least 9 or at least 10 nucleobases in length, such as at least 12 nucleobases or at least 14 nucleobases in length, such as 14 or 16 nucleobases in length.
9. The compound according to any one of the preceding embodiments, wherein said nucleic acid which encodes said mammalian PCSK9 is SEQ ID NO 80 or naturally occurring variant thereof.

10. The compound according to any one of the preceding embodiments, wherein said compound consists of no more than 22 nucleobases, such as no more than 20 nucleobases, such as no more than 18 nucleobases, optionally conjugated with one or more non-nucleobase compounds.

11. The compound according to embodiment 10 wherein said compound consists of either 13, 14, 15, 16 or 17 nucleobases, optionally conjugated with one or more non-nucleobase compounds.
12. The compound according to any one of the preceding embodiments wherein said compound comprises of no more than 3 mismatches with the corresponding region of the nucleic acid which encodes said mammalian PCSK9.
13. The compound according to any one of the preceding embodiments, wherein said subsequence or said combined contiguous nucleobase sequence corresponds to a sequence present in a nucleic acid sequence selected from the group consisting of SEQ ID's 1-79 and SEQ ID's 81-94, or a sequence present in table 2.
14. The compound according to embodiment 13, wherein said subsequence corresponds to a sequence present in a nucleic acid sequence selected from the group consisting of SEQ ID NO's 4-5, SEQ ID NO 13, SEQ ID No's 21-22, SEQ ID NO 25, SEQ ID NO's 39-44, SEQ ID NO's 59-60, SEQ ID NO 67, SEQ ID NO's 69-70 and SEQ ID NO's 76-79, or wherein said subsequence corresponds to any one of Cpd ID #'s 1-32.
15. The compound according to any one of the preceding embodiments which is an antisense oligonucleotide.
16. The compound according to embodiment 15, wherein the antisense oligonucleotide consists of a combined total of between 12 and 25 nucleobases, wherein the nucleobase sequence of said oligonucleotide is at least 80% homologous, such as at least 85% homologous, such as at least 90% homologous, such as at least 95% homologous, such as at least 97% homologous, such as 100% homologous to the reverse complement of a corresponding region of the nucleic acid which encodes said mammalian PCSK9.
17. The compound according to any one of the preceding embodiments, wherein said compound, said subsequence, said combined contiguous nucleobase sequence and/or said flanking sequence or sequences, comprise at least one nucleotide analogue.
18. The compound according to embodiment 17, wherein said compound, said subsequence, said combined contiguous nucleobase sequence and/or said flanking sequence or sequences comprise a total of between 2 and 10 nucleotide analogues, such as between 5 and 8 nucleotide analogues.
19. The compound according to any one of the preceding embodiments, wherein the antisense oligonucleotide is a gapmer, a headmer, a tailmer or a mixmer, which comprises nucleobases which are both nucleotides and nucleotide analogues.
20. The compound according to embodiment 19, wherein said compound, said sub-sequence, or said combined contiguous nucleobase sequence is a gapmer of formula, in 5′ to 3′ direction, A-B-C, and optionally of formula A-B-C-D, wherein:
A consists or comprises of at least one nucleotide analogue, such as between 1-6 nucleotide analogues, preferably between 2-5 nucleotide analogues, preferably 2, 3 or 4 nucleotide analogues, such as 3 or 4 consecutive nucleotide analogues and;
B consists or comprises at least five consecutive nucleobases which are capable of recruiting RNAseH, such as between 1 and 12, or between 6-10, or between 7-9, such as 8 consecutive nucleobases which are capable of recruiting RNAseH, and;
C consists or comprises of at least one nucleotide analogue, such as between 1-6 nucleotide analogues, preferably between 2-5 nucleotide analogues, preferably 2, 3 or 4 nucleotide analogues, such as 3 or 4 consecutive nucleotide analogues and;
D where present, consists or comprises, preferably consists, of one or more DNA nucleotide, such as between 1-3 or 1-2 DNA nucleotides.
   21. The compound according to embodiment 20, wherein:
A Consists of 3 or 4 consecutive nucleotide analogues;
B Consists of 8 or 9 or 10 consecutive DNA nucleotides or equivalent nucleobases which are capable of recruiting RNAseH;
C Consists of 3 or 4 consecutive nucleotide analogues;
D Consists, where present, of one DNA nucleotide.
   22. The compound according to embodiment 20, wherein:
A Consists of 3 consecutive nucleotide analogues;
B Consists of 9 consecutive DNA nucleotides or equivalent nucleobases which are capable of recruiting RNAseH;
C Consists of 3 consecutive nucleotide analogues;
D Consists, where present, of one DNA nucleotide.
   23. A compound according to embodiment 20, wherein:
A Consists of 3 consecutive nucleotide analogues; B Consists of 10 consecutive DNA nucleotides or equivalent nucleobases which are capable of recruiting RNAseH;
C Consists of 3 consecutive nucleotide analogues;
D Consists, where present, of one DNA nucleotide.
   24. The compound according to embodiments 20-23, wherein regions A and C correspond to said 5′ and said 3′ flanking regions, and region B corresponds to said sub-sequence.
   25. The compound according to anyone of embodiments 20-24, wherein B comprises or consists of DNA nucleobases.
   26. The compound according to any one of embodiments 17-25, wherein at least one nucleotide analogue is a Locked Nucleic Acid (LNA) unit.
   27. The compound according to embodiment 26, which comprise between 1 and 10 LNA units such as between 2 and 8 nucleotide LNA units.
   28. The compound according to embodiment 27 where all the nucleotide analogues present in said compound are LNA units.
   29. The compound according to any one of the embodiments 26-28, wherein the LNAs are independently selected from oxy-LNA, thio-LNA, and amino-LNA, in either of the D-β and L-α configurations or combinations thereof.
   30. The compound according to embodiment 29, wherein the LNAs are all β-D-oxy-LNA.
   31. The compound according to any one of the preceding embodiments, wherein at least one of the nucleobases present in the nucleotides or nucleotide analogues is a modified nucleobase selected from the group consisting of 5-methylcytosine, isocytosine, pseudoisocytosine, 5-bromouracil, 5-propynyluracil, 6-aminopurine, 2-aminopurine, inosine, diaminopurine, and 2-chloro-6-aminopurine.
   32. The compound according to any one of the preceding embodiments, wherein said compound hybridises with a corresponding mammalian PCSK9 mRNA with a Tₘ of at least 50° C.
   33. The compound according to any one of the preceding embodiments, wherein said compound hybridises with a corresponding mammalian PCSK9 mRNA with a Tₘ of no greater than 80° C.
   34. The compound according to any one of the preceding embodiments, where the nucleobase sequence consists or comprises of a sequence which is, or corresponds to, a sequence selected from the group consisting of SEQ ID NO's 1-79 and SEQ ID's 81-94, or a sequence present in table 2, wherein the nucleotides present in the compound may be substituted with a corresponding nucleotide analogue such as LNA, which are independently selected from oxy-LNA, thio-LNA, and amino-LNA, in either of the D-β and L-α configurations or combinations thereof and wherein said compound may comprise one, two, or three mismatches against said selected sequence, and optionally, linkage groups other than phosphorothioate may be used.
   35. The compound according to embodiment 34 which consists of a sequence selected from the group consisting of SEQ ID NOS 1-79 and SEQ ID's 81-94, or a sequence present in table 2, wherein the oligonucleotides are 12mers of 2-8-2 design or 14mers of 3-8-3 design, wherein the flanking sequences are LNA nucleotides which are independently selected from oxy-LNA, thio-LNA, and amino-LNA, in either of the D-β and L-α configurations or combinations thereof.
   36. The compound according to any one of embodiments 34-35, wherein the sequence is selected from any one of SEQ ID NO's 4-5, SEQ ID NO 13, SEQ ID No's 21-22, SEQ ID NO 25, SEQ ID NO's 39-44, SEQ ID NO's 59-60, SEQ ID NO 67, SEQ ID NO's 69-70 and SEQ ID NO's 76-79, or which is selected from any one of Cpd ID #'s 1-32.
   37. A conjugate comprising the compound according to any one of the embodiments 1-36 and at least one non-nucleotide or non-polynucleotide moiety covalently attached to said compound
   38. A pharmaceutical composition comprising a compound as defined in any of embodiments 1-36 or a conjugate as defined in embodiment 37, and a pharmaceutically acceptable diluent, carrier, salt or adjuvant
   39. A pharmaceutical composition according to 38, wherein the compound is constituted as a pro-drug.
   40. A pharmaceutical composition according to any one of embodiments 38-39, which further comprises an anti-inflamatory compounds and/or antiviral compounds.
   41. The pharmaceutical composition according to embodiment 40 further comprising at least one further agent which is capable of lowering blood serum cholesterol.
   42. The pharmaceutical composition according to embodiment 41, wherein the at least one further agent is a statin or a fibrogen.
   43. The pharmaceutical composition according to embodiment 41 or 42, wherein the at least one further agent is a modulator of Apolipoprotein B-100 (Apo-B).
   44. The pharmaceutical composition according to embodiment 43, wherein the modulator of Apo-B is an antisense oligonucleotide.
   45. Use of a compound as defined in any one of the embodiments 1-36, or a conjugate as defined in embodiment 37, for the manufacture of a medicament for the treatment of hypercholesterolemia or a related disorder.
   46. A method for treating hypercholesterolemia or related disorder, said method comprising administering a compound as defined in one of the embodiments 1-36, or a conjugate as defined in embodiment 37, or a pharmaceutical composition as defined in any one of the embodiments 38-44, to a patient in need thereof.
   47. A method of inhibiting the expression of PCSK9 in a cell or a tissue, the method comprising the step of contacting said cell or tissue with a compound as defined in one of the embodiments 1-36, or a conjugate as defined in embodiment 37, or a pharmaceutical composition as defined in any one of the embodiments 38-44, so that expression of PCSK9 is inhibited.
   48. A method of modulating expression of a PCSK9 gene comprising contacting the gene or RNA from the gene with the compound as defined in one of the embodiments 1-36, or a conjugate as defined in embodiment 37, or a pharmaceutical composition as defined in any one of the embodiments 38-44, so that gene expression is modulated.
   49. A method of modulating the level of blood serum cholesterol in a mammal, the method comprising the step of contacting said cell or tissue with a compound as defined in one of the embodiments 1-36, or a conjugate as defined in embodiment 37, or a pharmaceutical composition as defined in any one of the embodiments 38-44, so that the blood serum cholesterol level is modulated.
   50. A pharmaceutical composition comprising a non-toxic dosage of any one of Cpd ID # 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 or 32.
   51. Compound ID # 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 or 32 for use as a medicament.

| | SEQ | | SEQ |
|---|---|---|---|
| Sequence | ID # | Sequence | ID # |
| 5′-AGGCTAGCACCAG-3′ | 1 | 5′-AGAAAGCTAAGCC-3′ | 47 |
| 5′-CAAGGCTAGCACC-3′ | 2 | 5′-GCTAGATGCCATC-3′ | 48 |
| 5′-GCAAGGCTAGCAC-3′ | 3 | 5′-GCTAGATGCCAT-3′ | 49 |
| 5′-ACGCAAGGCTAG-3′ | 4 | 5′-GGCTAGATGCCAT-3′ | 50 |
| 5′-AACGCAAGGCTA-3′ | 5 | 5′-CAGAGTAAAGGTG-3′ | 51 |
| 5′-GATCCTTGGCGC-3′ | 6 | 5′-GTTGCTAGCACAG-3′ | 52 |
| 5′-TGGTGAGGTATCC-3′ | 7 | 5′-TGTTGCTAGCACA-3′ | 53 |
| 5′-CATGCAGGATCTT-3′ | 8 | 5′-GTCCTAGGTGATG-3′ | 54 |
| 5′-GACATGCAGGATC-3′ | 9 | 5′-AGTCCTAGGTGAT-3′ | 55 |
| 5′-CATGACCCTGCCC-3′ | 10 | 5′-CAGTCCTAGGTGA-3′ | 56 |
| 5′-TGACCATGACCCT-3′ | 11 | 5′-ACTGCACACTGCC-3′ | 57 |
| 5′-ATGCTGGCACCCT-3′ | 12 | 5′-GTTGGCTGAGACA-3′ | 58 |
| 5′-GCTGTACCCACCC-3′ | 13 | 5′-GCGAATGTGTAC-3′ | 59 |
| 5′-TGGAGGCACCAAT-3′ | 14 | 5′-GCCCAATCTGCG-3′ | 60 |
| 5′-CTCTGTGACACAA-3′ | 15 | 5′-CCTCACTGTTACC-3′ | 61 |
| 5′-GTCCTGCAAAAC-3′ | 16 | 5′-GCCTCACTGTTAC-3′ | 62 |
| 5′-CAGACCAGCTTGC-3′ | 17 | 5′-AGCCTCACTGTTA-3′ | 63 |
| 5′-GCAGACCAGCTTG-3′ | 18 | 5′-GCCTTAGAAGCAT-3′ | 64 |
| 5′-TCCCAGTGGGAGC-3′ | 19 | 5′-TGCCTTAGAAGCA-3′ | 65 |
| 5′-ATGCTGGCCTCCC-3′ | 20 | 5′-ACCATGCCTTAGA-3′ | 66 |
| 5′-GTGTTGTCTACG-3′ | 21 | 5′-ACCATGCCTTAG-3′ | 67 |
| 5′-ACGTGTTGTCTA-3′ | 22 | 5′-GACCATGCCTTAG-3′ | 68 |
| 5′-CAGACACCCATCC-3′ | 23 | 5′-GACCATGCCTTA-3′ | 69 |
| 5′-AGCCCTTGACCCT-3′ | 24 | 5′-CCGACCATGCCT-3′ | 70 |
| 5′-CGGAACCATTTT-3′ | 25 | 5′-TGCTTGCTTGGGT-3′ | 71 |
| 5′-GAGTGAGTGAGTT-3′ | 26 | 5′-AAGTTGGCTGTAA-3′ | 72 |
| 5′-AATGGTGAAATGC-3′ | 27 | 5′-ACAGGTCTAGAAA-3′ | 73 |
| 5′-AGTCATTCTGCCC-3′ | 28 | 5′-AACAGGTCTAGAA-3′ | 74 |
| 5′-TTGAATGCCTGGC-3′ | 29 | 5′-CCAGAATAAATAT-3′ | 75 |
| 5′-ATTGAATGCCTGG-3′ | 30 | 5′-ACTGTGATGACCTC-3′ | 76 |
| 5′-GATTGAATGCCTG-3′ | 31 | 5′-TAATCAGGGAGCCC-3′ | 77 |
| 5′-GGATTGAATGCCT-3′ | 32 | 5′-TTAATCAGGGAGCC-3′ | 78 |
| 5′-GACCTGAGGATTG-3′ | 33 | 5′-TCGGGTGCTTCG-3′ | 79 |
| 5′-GGTGGAGACCTGA-3′ | 34 | 5′-AGACAGAGGAGTC-3′ | 85 |
| 5′-CATGGGAAGAATC-3′ | 35 | 5′-AGACAAAGGAGTC-3′ | 87 |
| 5′-CCCTATCCATGGG-3′ | 36 | 5′-CCCAGAGTGAGTG-3′ | 87 |
| 5′-TGTTTGTCCCTGC-3′ | 37 | 5′-CCCAGAGTGAGGG-3′ | 88 |
| 5′-ATGTTTGTCCCTG-3′ | 38 | 5′-CGGCTGTACCCAC-3′ | 89 |
| 5′-CGATGTTTGTCC-3′ | 39 | 5′-CGGCTATACCCAC-3′ | 90 |
| 5′-ATTAATCAGGGAG-3′ | 40 | 5′-CCTTGACTTTGCA-3′ | 91 |
| 5′-ATTAATCAGGGA-3′ | 41 | 5′-CCTTGATTTTGCA-3′ | 92 |
| 5′-CATTAATCAGGGA-3′ | 42 | 5′-ATCGTCCCGGAA-3′ | 93 |
| 5′-TCCATTAATCAGG-3′ | 43 | 5′-GTCGTCCCGGAA-3′ | 94 |
| 5′-CTCCATTAATCAG-3′ | 44 | | |
| 5′-CCTCCATTAATCA-3′ | 45 | | |
| 5′-GCCTCCATTAATC-3′ | 46 | | |

Table 2: sequence of SEQ ID NO′s 1-79. Preferred designs of specific compounds/
LNA antisense oligonucleotides are 12 mers, 2-8-2 (LNA-DNA-LNA), 13 mers,
2-8-3 or 3-8-2 (LNA-DNA-LNA) and 14 mers 3-8-3 (LNA-DNA-LNA). The above
table provides for each sequence, the SEQ ID used in the sequence listing.

### EXAMPLES

LNA monomer and oligonucleotide synthesis were performed using the methodology referred to in Examples 1 and 2 of PCT/EP2007/060703.

The stability of LNA oligonucleotides in human or rat plasma is performed using the methodology referred to in Example 4 of PCT/EP2007/060703

The treatment of in vitro cells with LNA anti-PCSK9 antisense oligonucleotides is performed using the methodology referred to in Examples 5 and 6 of PCT/EP2007/060703

The analysis of Oligonucleotide Inhibition of PCSK9 expression by PCSK9 specific quantitative PCR in both an in vitro and in vivo model is performed using the methodology referred to in example 7 and 8 of PCT/EP2007/060703.

In vitro analysis of dose response in cell culture of LNA antisense inhibition of Human and murine PCSK9 expression is performed using the methodology referred to in examples 9 and 10 of PCT/EP2007/060703 respectively.

Testing of cholesterol levels in mouse serum, LDL-receptor protein level in mouse liver, lipoprotein class composition in serum, is performed using the methodology referred to in examples 11-13 of PCT/EP2007/060703 respectively.

In vivo experiments using oligomers of the invention targeting PCSK9 and subsequent analysis are performed using the methods disclosed in examples 14-17 of PCT/EP2007/060703.

The above mentioned examples of PCT/EP2007/060703 are hereby specifically incorporated by reference.

### Example 1

Design of the Oligonucleotide

In a specific preferred design of the oligonucleotides of the invention, oligomers comprising 12 nucleotide sequences of Table 2 are designed as 2-8-2 (LNA-DNA-LNA) oligomers, oligomers comprising 13 nucleotide sequences of Table 2 are designed as 3-8-2, or 2-8-3 (LNA-DNA-LNA) oligomers and oligomers comprising 14 nucleotide sequences of Table 2 are designed as 3-8-3 (LNA-DNA-LNA) oligomers, wherein the LNAs are independently selected from oxy-LNA, thio-LNA, and amino-LNA, in either of the D-β and L-α configurations or combinations thereof.

TABLE 3

| | | Corres- | |
|---|---|---|---|
| Com- | | ponding | |
| pound | | SEQ ID | |
| # | Sequence | # | Length |
| 15 | 5′-ACgcaaggctAG-3′ | 4 | 12 |
| 14 | 5′-AAcgcaaggcTA-3′ | 5 | 12 |
| 17 | 5′-GCTgtacccacCC-3′ | 13 | 13 |
| 9 | 5′-GTgttgtctaCG-3′ | 21 | 12 |
| 8 | 5′-ACgtgttgtcTA-3′ | 22 | 12 |
| 7 | 5′-CGaaccattTT-3′ | 25 | 12 |
| 6 | 5′-CGatgtttgtCC-3′ | 39 | 12 |
| 18 | 5′-ATTaatcagggAG-3′ | 40 | 13 |
| 19 | 5′-ATtaatcaggGA-3′ | 41 | 12 |
| 20 | 5′-CATtaatcaggGA-3′ | 42 | 13 |
| 21 | 5′-TCCattaatcaGG-3′ | 43 | 13 |
| 22 | 5′-CTCcattaatcAG-3′ | 44 | 13 |
| 5 | 5′-GCgaatgtgtAC-3′ | 59 | 12 |
| 4 | 5′-GCccaatctgCG-3′ | 60 | 12 |
| 3 | 5′-ACcatgccttAG-3′ | 67 | 12 |
| 2 | 5′-GAccatgccTA-3′ | 69 | 12 |
| 1 | 5′-CCgaccatgcCT-3′ | 70 | 12 |
| 12 | 5′-ACTgtgatgacCTC-3′ | 76 | 14 |
| 11 | 5′-TAAtcagggagCCC-3′ | 77 | 14 |
| 10 | 5′-TTAatcagggaGCC-3′ | 78 | 14 |
| 13 | 5′-TCgggtgcttCG-3′ | 79 | 12 |
| 16 | 5′- [symbol] gₛtₛgₛaₛgₛtₛgₛaₛ [symbol] -3′ | 26 | 13 |
| 23 | 5′- [symbol] aₛcₛaₛgₛaₛgₛgₛaₛ [symbol] -3′ | 85 | 13 |
| 24 | 5′- [symbol] aₛcₛaₛaₛaₛgₛgₛaₛ [symbol] -3′ | 86 | 13 |
| 25 | 5′- [symbol] cₛaₛgₛaₛgₛtₛgₛaₛ [symbol] -3′ | 87 | 13 |
| 26 | 5′- [symbol] cₛaₛgₛaₛgₛtₛgₛaₛ [symbol] -3′ | 88 | 13 |
| 27 | 5′- [symbol] gₛcₛtₛgₛtₛaₛcₛcₛ [symbol] -3′ | 89 | 13 |
| 28 | 5′- [symbol] gₛcₛtₛaₛtₛaₛcₛcₛ [symbol] -3′ | 90 | 13 |
| 29 | 5′- [symbol] tₛtₛgₛaₛcₛtₛtₛtₛ [symbol] -3′ | 91 | 13 |
| 30 | 5′- [symbol] tₛtₛgₛaₛtₛtₛtₛtₛ [symbol] -3′ | 92 | 13 |
| 31 | 5′- [symbol] cₛgₛtₛcₛcₛcₛgₛgₛ [symbol] -3′ | 93 | 12 |
| 32 | 5′- [symbol] cₛgₛtₛcₛcₛcₛgₛgₛ [symbol] -3′ | 94 | 12 |

Table 3
Capitals are LNA nucleotides
Small letters are DNA nucleotides
13 mers are shown as 3-8-2 (LNA-DNA-LNA), but in an equally preferred
embodiment, 12 mers are 2-8-2 (LNA-DNA-LNA).
In a preferred embodiment, internucleoside bonds are fully thiolated.
ₛare Phosphothioate internucleotide bonds.
^{o}indicate oxy LNA, such as beta-D-oxy-LNA
^{m}indicate 5′methylation (in connection with cytosines)

### Example 2

### In vitro Model

### Cell Culture

The effect of antisense compounds on target nucleic acid expression can be tested in any of a variety of cell types provided that the target nucleic acid is present at measurable levels. Target can be expressed endogenously or by transient or stable transfection of a nucleic acid encoding said nucleic acid.

The expression level of target nucleic acid can be routinely determined using, for example, Northern blot analysis, Quantitative PCR, Ribonuclease protection assays. The following cell types are provided for illustrative purposes, but other cell types can be routinely used, provided that the target is expressed in the cell type chosen.

Cells were cultured in the appropriate medium as described below and maintained at 37° C. at 95-98% humidity and 5% CO₂. Cells were routinely passaged 2-3 times weekly.

Huh-7: Human liver cell line Huh-7 was purchased from ATCC and cultured in Eagle MEM (Sigma) with 10% FBS+Glutamax I+non-essential amino acids+gentamicin.

### Example 3

### In vitro Model

### Treatment with Antisense Oligonucleotide

Cell culturing and transfection: Huh-7 and Hepa 1-6 cells were seeded in 6-well plates at 37° C. (5% CO₂) in growth media supplemented with 10% FBS, Glutamax I and Gentamicin. When the cells were 60-70% confluent, they were transfected in duplicates with different concentrations of oligonucleotides (0.04-25 nM) using Lipofectamine 2000 (5 μg/mL). Transfections were carried out essentially as described by Dean et al. (1994, 313C 269:16416-16424). In short, cells were incubated for 10 min. with Lipofectamine in OptiMEM followed by addition of oligonucleotide to a total volume of 0.5 mL transfection mix per well. After 4 hours, the transfection mix was removed, cells were washed and grown at 37° C. for approximately 20 hours (mRNA analysis and protein analysis in the appropriate growth medium. Cells were then harvested for protein and RNA analysis.

### Example 4

### In vitro Model: Extraction of RNA and cDNA Synthesis

### Total RNA Isolation

Total RNA was isolated using RNeasy mini kit (Qiagen). Cells were washed with PBS, and Cell Lysis Buffer (RTL, Qiagen) supplemented with 1% mercaptoethanol was added directly to the wells. After a few minutes, the samples were processed according to manufacturer's instructions.

### First Strand Synthesis

First strand synthesis was performed using either OmniScript Reverse Transcriptase kit or M-MLV Reverse transcriptase (essentially as described by manufacturer (Ambion)) according to the manufacturer's instructions (Qiagen). When using OmniScript Reverse Transcriptase 0.5 μg total RNA each sample, was adjusted to 12 μl and mixed with 0.2 μl poly (dT)₁₂₋₁₈ (0.5 μg/μl) (Life Technologies), 2 μl dNTP mix (5 mM each), 2 μl 10×RT buffer, 0.5 μl RNAguard™ RNase Inhibitor (33 units/mL, Amersham) and 1 μl OmniScript Reverse Transcriptase followed by incubation at 37° C. for 60 min. and heat inactivation at 93° C. for 5 min.

When first strand synthesis was performed using random decamers and M-MLV-Reverse Transcriptase (essentially as described by manufacturer (Ambion)) 0.25 μg total RNA of each sample was adjusted to 10.8 μl in H₂O. 2 μl decamers and 2 μl dNTP mix (2.5 mM each) was added. Samples were heated to 70° C. for 3 min. and cooled immediately in ice water and added 3.25 μl of a mix containing (2 μl 10×RT buffer; 1 μl M-MLV Reverse Transcriptase; 0.25 μl RNAase inhibitor). cDNA is synthesized at 42° C. for 60 min followed by heating inactivation step at 95° C. for 10 min and finally cooled to 4° C.

### Example 5

### In Vitro and In vivo Model: Analysis of Oligonucleotide Inhibition of PCSK9 Expression by Real-Time PCR

Antisense modulation of PCSK9 expression can be assayed in a variety of ways known in the art. For example, PCSK9 mRNA levels can be quantitated by, e.g., Northern blot analysis, competitive polymerase chain reaction (PCR), or real-time PCR. Real-time quantitative PCR is presently preferred. RNA analysis can be performed on total cellular RNA or mRNA.

Methods of RNA isolation and RNA analysis such as Northern blot analysis is routine in the art and is taught in, for example, Current Protocols in Molecular Biology, John Wiley and Sons.

Real-time quantitative (PCR) can be conveniently accomplished using the commercially iQ Multi-Color Real Time PCR Detection System available from BioRAD. Real-time Quantitative PCR is a technique well known in the art and is taught in for example Heid et al. Real time quantitative PCR, Genome Research (1996), 6: 986-994.

Real-Time Quantitative PCR Analysis of PCSK9 mRNA Levels

To determine the relative human PCSK9 mRNA level in treated and untreated samples, the generated cDNA was used in quantitative PCR analysis using an iCycler from Bio-Rad or 7500 Fast Real-Time PCR System from Applied Biosystems.

8 μl of 10-fold diluted cDNA was added 52 μl of a mix containing 29.5 μl Platinum qPCR Supermix-UDG (Invitrogen) 19.2 μl H₂O and 3.0 μl of a 20× human PCSK9 or GAPDH TaqMan gene expression assay (Applied Biosystems). Each sample was analysed in duplicates. PCR program: 95° C. for 20 seconds followed by 40 cycles of 95° C., 3 seconds, 60° C., 30 seconds.

Mouse PCSK9: Mouse PCSK9 expression is quantified using a mouse PCSK9 or GAPDH TaqMan gene expression assay (Applied Biosystems) 8 μl of 10-fold diluted cDNA is added 52 μl of a mix containing 29.5 μl Platinum qPCR Supermix-UDG (Invitrogen) 19.2 μl H₂O and 3.0 μl of a 20× mouse PCSK9 or GAPDH TaqMan gene expression assay (Applied Biosystems). Each sample is analysed in duplicates. PCR program: 95° C. for 20 seconds followed by 40 cycles of 95° C., 3 seconds, 60° C., 30 seconds.

PCSK9 mRNA expression is normalized to mouse Gapdh mRNA which was similarly quantified using Q-PCR.

2-fold dilutions of cDNA synthesised from untreated human Hepatocyte cell line (Huh-7) (diluted 5 fold and expressing both PCSK9 and Gapdh) is used to prepare standard curves for the assays. Relative quantities of PCSK9 mRNA were determined from the calculated Threshold cycle using the iCycler iQ Real Time Detection System software.

### Example 6

### In vitro Analysis: Dose Response in Cell Culture (Human Hepatocyte Huh-7)/Antisense Inhibition of Human PCSK9 Expression

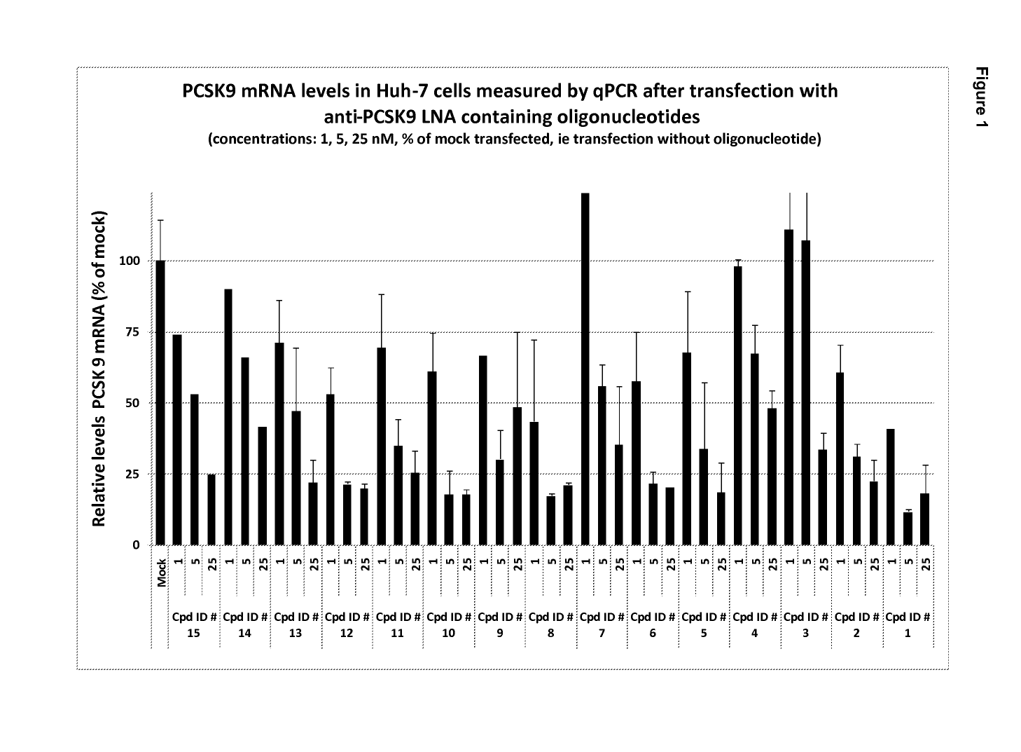

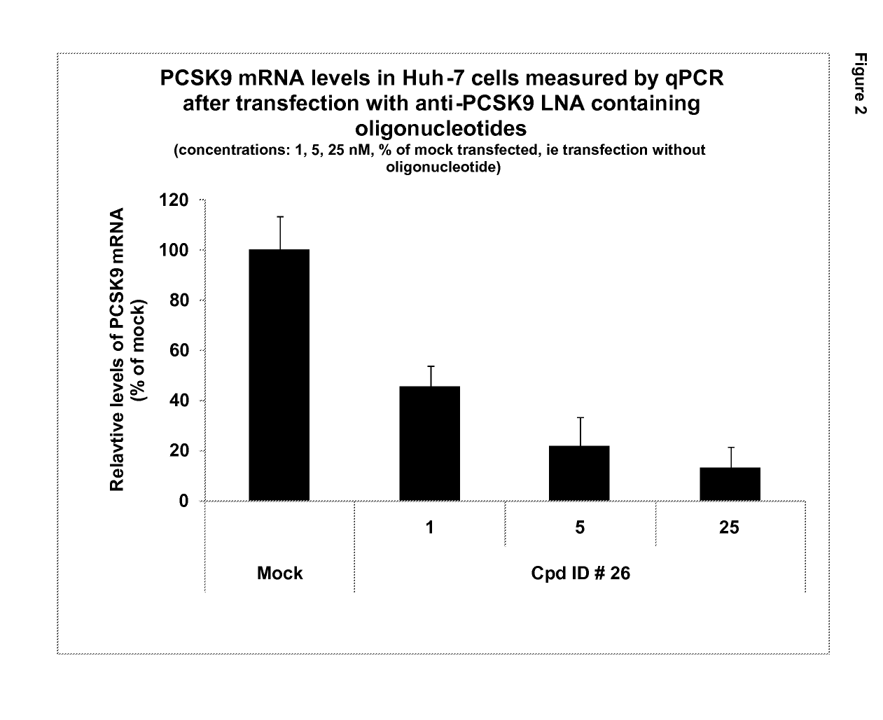

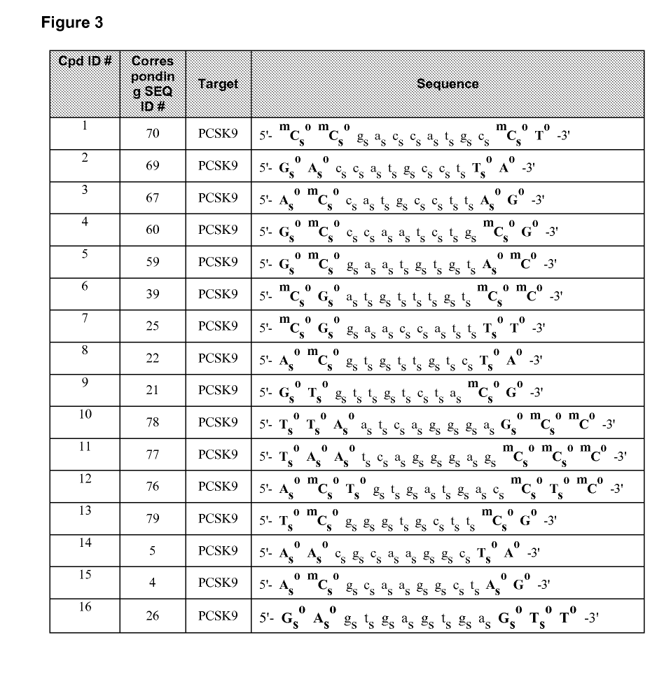

In accordance with the present invention, a series of oligonucleotides were designed to target different regions of the human PCSK9 mRNA. See Table 3 and FIG. 3. Oligonucleotide compounds were evaluated for their potential to knockdown PCSK9 mRNA in Human hepatocytes (Huh-7 cells) following lipid-assisted uptake of Compound ID NO#s: 1-15 (FIG. 1) and Cpd ID # 16 (FIG. 2). The experiment was performed as described in examples 2-5. The results showed very potent down regulation (50 to ≧80%) with 25 nM for all compounds.

1. An oligomer of between 10-30 nucleotides in length which comprises a contiguous nucleotide sequence of a total of between 10-30 nucleotides, wherein said contiguous nucleotide sequence is at least 80% homologous to a region corresponding to a mammalian PCSK9 gene or the reverse complement of an mRNA, such as SEQ ID NO: 80 or naturally occurring variant thereof, wherein the contiguous nucleotide sequence is at least 80% homologous to a region corresponding to a sequence selected from the group consisting of SEQ ID NO: 1-79 and SEQ ID NO: 81-94.

2. The oligomer according to claim 1, wherein the contiguous nucleotide sequence comprises no mismatches or no more than one or two mismatches with the reverse complement of the corresponding region of SEQ ID NO: 80.

3. The oligomer according to any one of claims 1-2, wherein the nucleotide sequence of the oligomer consists of the contiguous nucleotide sequence.

4. The oligomer according to any one of claims 1-3, wherein the contiguous nucleotide sequence is between 10-18 nucleotides in length.

5. The oligomer according to any one of claims 1-4, wherein the contiguous nucleotide sequence comprises nucleotide analogues.

6. The oligomer according to claim 5, wherein the nucleotide analogues are sugar modified nucleotides, such as sugar modified nucleotides selected from the group consisting of: Locked Nucleic Acid (LNA) units; 2′-O-alkyl-RNA units, 2′-OMe-RNA units, 2′-amino-DNA units, and 2′-fluoro-DNA units.

7. The oligomer according to claim 5, wherein the nucleotide analogues are LNA.

8. The oligomer according to any one of claims 5-7 which is a gapmer.

9. The oligomer according to any one of claims 5-8, wherein the oligomer is any one of Cpd ID #'s 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 or 32.

10. The oligomer according to any one of claims 1-9, which inhibits the expression of PCSK9 gene or mRNA in a cell which is expressing PCSK9 gene or mRNA.

11. The oligomer according to any one of claims 1-10, wherein the oligomer sequence comprises one of the sequences selected from the group consisting of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 81, 82, 83, 84, 85, 86, 87, 88, 89, 90, 91, 92, 93, 94, or a sub-sequence of at least 10 contiguous nucleotides thereof, wherein said oligomer (or contiguous nucleotide portion thereof) may optionally comprise one, two, or three mismatches against said selected sequence.

12. A conjugate comprising the oligomer according to any one of claims 1-11, and at least one non-nucleotide or non-polynucleotide moiety covalently attached to said oligomer.

13. A pharmaceutical composition comprising the oligomer according to any one of claims 1-11, or the conjugate according to claim 12, and a pharmaceutically acceptable diluent, carrier, salt or adjuvant.

14. The oligomer according to any one of claims 1-11, or the conjugate according to claim 12, for use as a medicament, such as for the treatment of HYPERCHOLESTEROLEMIA AND RELATED DISORDERS.

15. The use of an oligomer according to any one of the claims 1-11, or a conjugate as defined in claim 12, for the manufacture of a medicament for the treatment of HYPERCHOLESTEROLEMIA AND RELATED DISORDERS.

16. A method of treating HYPERCHOLESTEROLEMIA AND RELATED DISORDERS, said method comprising administering an oligomer according to any one of the claims 1-11, or a conjugate according to claim 12, or a pharmaceutical composition according to claim 13, to a patient suffering from, or likely to suffer from HYPERCHOLESTEROLEMIA AND RELATED DISORDERS.

17. A method for the inhibition of PCSK9 in a cell which is expressing PCSK9, said method comprising administering an oligomer according to any one of the claims 1-11, or a conjugate according to claim 12 to said cell so as to inhibit PCSK9 in said cell.


SEQUENCE LISTING (94 sequences)

SEQ ID NO: 1 - DNA, 13 bp, artificial sequence
  synthetically generated oligonucleotide
aggctagcaccag

SEQ ID NO: 2 - DNA, 13 bp, artificial sequence
  synthetically generated oligonucleotide
caaggctagcacc

SEQ ID NO: 3 - DNA, 13 bp, artificial sequence
  synthetically generated oligonucleotide
gcaaggctagcac

SEQ ID NO: 4 - DNA, 12 bp, artificial sequence
  synthetically generated oligonucleotide
acgcaaggctag

SEQ ID NO: 5 - DNA, 12 bp, artificial sequence
  synthetically generated oligonucleotide
aacgcaaggcta

SEQ ID NO: 6 - DNA, 12 bp, artificial sequence
  synthetically generated oligonucleotide
gatccttggcgc

SEQ ID NO: 7 - DNA, 13 bp, artificial sequence
  synthetically generated oligonucleotide
tggtgaggtatcc

SEQ ID NO: 8 - DNA, 13 bp, artificial sequence
  synthetically generated oligonucleotides
catgcaggatctt

SEQ ID NO: 9 - DNA, 13 bp, artificial sequence
  synthetically generated oligonucleotide
gacatgcaggatc

SEQ ID NO: 10 - DNA, 13 bp, artificial sequence
  synthetically generated oligonucleotide
catgaccctgccc

SEQ ID NO: 11 - DNA, 13 bp, artificial sequence
  synthetically generated oligonucleotide
tgaccatgaccct

SEQ ID NO: 12 - DNA, 13 bp, artificial sequence
  synthetically generated oligonucleotide
atgctggcaccct

SEQ ID NO: 13 - DNA, 13 bp, artificial sequence
  synthetically generated oligonucleotide
gctgtacccaccc

SEQ ID NO: 14 - DNA, 13 bp, artificial sequence
  synthetically generated oligonucleotide
tggaggcaccaat

SEQ ID NO: 15 - DNA, 13 bp, artificial sequence
  synthetically generated oligonucleotide
ctctgtgacacaa

SEQ ID NO: 16 - DNA, 12 bp, artificial sequence
  synthetically generated oligonucleotide
gtcctgcaaaac

SEQ ID NO: 17 - DNA, 13 bp, artificial sequence
  synthetically generated oligonucleotide
cagaccagcttgc

SEQ ID NO: 18 - DNA, 13 bp, artificial sequence
  synthetically generated oligonucleotide
gcagaccagcttg

SEQ ID NO: 19 - DNA, 13 bp, artificial sequence
  synthetically generated oligonucleotide
tcccagtgggagc

SEQ ID NO: 20 - DNA, 13 bp, artificial sequence
  synthetically generated oligonucleotide
atgctggcctccc

SEQ ID NO: 21 - DNA, 12 bp, artificial sequence
  synthetically generated oligonucleotide
gtgttgtctacg

SEQ ID NO: 22 - DNA, 12 bp, artificial sequence
  synthetically generated oligonucleotide
acgtgttgtcta

SEQ ID NO: 23 - DNA, 13 bp, artificial sequence
  synthetically generated oligonucleotide
cagacacccatcc

SEQ ID NO: 24 - DNA, 13 bp, artificial sequence
  synthetically generated oligonucleotide
agcccttgaccct

SEQ ID NO: 25 - DNA, 12 bp, artificial sequence
  synthetically generated oligonucleotide
cggaaccatttt

SEQ ID NO: 26 - DNA, 13 bp, artificial sequence
  synthetically generated oligonucleotide
gagtgagtgagtt

SEQ ID NO: 27 - DNA, 13 bp, artificial sequence
  synthetically generated oligonucleotide
aatggtgaaatgc

SEQ ID NO: 28 - DNA, 13 bp, artificial sequence
  synthetically generated oligonucleotide
agtcattctgccc

SEQ ID NO: 29 - DNA, 13 bp, artificial sequence
  synthetically generated oligonucleotide
ttgaatgcctggc

SEQ ID NO: 30 - DNA, 13 bp, artificial sequence
  synthetically generated oligonucleotide
attgaatgcctgg

SEQ ID NO: 31 - DNA, 13 bp, artificial sequence
  synthetically generated oligonucleotide
gattgaatgcctg

SEQ ID NO: 32 - DNA, 13 bp, artificial sequence
  synthetically generated oligonucleotide
ggattgaatgcct

SEQ ID NO: 33 - DNA, 13 bp, artificial sequence
  synthetically generated oligonucleotide
gacctgaggattg

SEQ ID NO: 34 - DNA, 13 bp, artificial sequence
  synthetically generated oligonucleotide
ggtggagacctga

SEQ ID NO: 35 - DNA, 13 bp, artificial sequence
  synthetically generated oligonucleotide
catgggaagaatc

SEQ ID NO: 36 - DNA, 13 bp, artificial sequence
  synthetically generated oligonucleotide
ccctatccatggg

SEQ ID NO: 37 - DNA, 13 bp, artificial sequence
  synthetically generated oligonucleotide
tgtttgtccctgc

SEQ ID NO: 38 - DNA, 13 bp, artificial sequence
  synthetically generated oligonucleotide
atgtttgtccctg

SEQ ID NO: 39 - DNA, 12 bp, artificial sequence
  synthetically generated oligonucleotide
cgatgtttgtcc

SEQ ID NO: 40 - DNA, 13 bp, artificial sequence
  synthetically generated oligonucleotide
attaatcagggag

SEQ ID NO: 41 - DNA, 12 bp, artificial sequence
  synthetically generated oligonucleotide
attaatcaggga

SEQ ID NO: 42 - DNA, 13 bp, artificial sequence
  synthetically generated oligonucleotide
cattaatcaggga

SEQ ID NO: 43 - DNA, 13 bp, artificial sequence
  synthetically generated oligonucleotide
tccattaatcagg

SEQ ID NO: 44 - DNA, 13 bp, artificial sequence
  synthetically generated oligonucleotide
ctccattaatcag

SEQ ID NO: 45 - DNA, 13 bp, artificial sequence
  synthetically generated oligonucleotide
cctccattaatca

SEQ ID NO: 46 - DNA, 13 bp, artificial sequence
  synthetically generated oligonucleotide
gcctccattaatc

SEQ ID NO: 47 - DNA, 13 bp, artificial sequence
  synthetically generated oligonucleotide
agaaagctaagcc

SEQ ID NO: 48 - DNA, 13 bp, artificial sequence
  synthetically generated oligonucleotide
gctagatgccatc

SEQ ID NO: 49 - DNA, 12 bp, artificial sequence
  synthetically generated oligonucleotide
gctagatgccat

SEQ ID NO: 50 - DNA, 13 bp, artificial sequence
  synthetically generated oligonucleotide
ggctagatgccat

SEQ ID NO: 51 - DNA, 13 bp, artificial sequence
  synthetically generated oligonucleotide
cagagtaaaggtg

SEQ ID NO: 52 - DNA, 13 bp, artificial sequence
  synthetically generated oligonucleotide
gttgctagcacag

SEQ ID NO: 53 - DNA, 13 bp, artificial sequence
  synthetically generated oligonucleotide
tgttgctagcaca

SEQ ID NO: 54 - DNA, 13 bp, artificial sequence
  synthetically generated oligonucleotide
gtcctaggtgatg

SEQ ID NO: 55 - DNA, 13 bp, artificial sequence
  synthetically generated oligonucleotide
agtcctaggtgat

SEQ ID NO: 56 - DNA, 13 bp, artificial sequence
  synthetically generated oligonucleotide
cagtcctaggtga

SEQ ID NO: 57 - DNA, 13 bp, artificial sequence
  synthetically generated oligonucleotide
actgcacactgcc

SEQ ID NO: 58 - DNA, 13 bp, artificial sequence
  synthetically generated oligonucleotide
gttggctgagaca

SEQ ID NO: 59 - DNA, 12 bp, artificial sequence
  synthetically generated oligonucleotide
gcgaatgtgtac

SEQ ID NO: 60 - DNA, 12 bp, artificial sequence
  synthetically generated oligonucleotide
gcccaatctgcg

SEQ ID NO: 61 - DNA, 13 bp, artificial sequence
  synthetically generated oligonucleotide
cctcactgttacc

SEQ ID NO: 62 - DNA, 13 bp, artificial sequence
  synthetically generated oligonucleotide
gcctcactgttac

SEQ ID NO: 63 - DNA, 13 bp, artificial sequence
  synthetically generated oligonucleotide
agcctcactgtta

SEQ ID NO: 64 - DNA, 13 bp, artificial sequence
  synthetically generated oligonucleotide
gccttagaagcat

SEQ ID NO: 65 - DNA, 13 bp, artificial sequence
  synthetically generated oligonucleotide
tgccttagaagca

SEQ ID NO: 66 - DNA, 13 bp, artificial sequence
  synthetically generated oligonucleotide
accatgccttaga

SEQ ID NO: 67 - DNA, 12 bp, artificial sequence
  synthetically generated oligonucleotide
accatgccttag

SEQ ID NO: 68 - DNA, 13 bp, artificial sequence
  synthetically generated oligonucleotide
gaccatgccttag

SEQ ID NO: 69 - DNA, 12 bp, artificial sequence
  synthetically generated oligonucleotide
gaccatgcctta

SEQ ID NO: 70 - DNA, 12 bp, artificial sequence
  synthetically generated oligonucleotide
ccgaccatgcct

SEQ ID NO: 71 - DNA, 13 bp, artificial sequence
  synthetically generated oligonucleotide
tgcttgcttgggt

SEQ ID NO: 72 - DNA, 13 bp, artificial sequence
  synthetically generated oligonucleotide
aagttggctgtaa

SEQ ID NO: 73 - DNA, 13 bp, artificial sequence
  synthetically generated oligonucleotide
acaggtctagaaa

SEQ ID NO: 74 - DNA, 13 bp, artificial sequence
  synthetically generated oligonucleotide
aacaggtctagaa

SEQ ID NO: 75 - DNA, 13 bp, artificial sequence
  synthetically generated oligonucleotide
ccagaataaatat

SEQ ID NO: 76 - DNA, 14 bp, artificial sequence
  synthetically generated oligonucleotide
actgtgatgacctc

SEQ ID NO: 77 - DNA, 14 bp, artificial sequence
  synthetically generated oligonucleotide
taatcagggagccc

SEQ ID NO: 78 - DNA, 14 bp, artificial sequence
  synthetically generated oligonucleotide
ttaatcagggagcc

SEQ ID NO: 79 - DNA, 12 bp, artificial sequence
  synthetically generated oligonucleotide
tcgggtgcttcg

SEQ ID NO: 80 - DNA, 3636 bp, homo sapiens
cagcgacgtcgaggcgctcatggttgcaggcgggcgccgccgttcagttcagggtctgagcctggaggagtgagccaggcagtgagactggctcgggcgggccgggacgcgtcgttgcagcagcggctcccagctcccagccaggattccgcgcgccccttcacgcgccctgctcctgaacttcagctcctgcacagtcctccccaccgcaaggctcaaggcgccgccggcgtggaccgcgcacggcctctaggtctcctcgccaggacagcaacctctcccctggccctcatgggcaccgtcagctccaggcggtcctggtggccgctgccactgctgctgctgctgctgctgctcctgggtcccgcgggcgcccgtgcgcaggaggacgaggacggcgactacgaggagctggtgctagccttgcgttccgaggaggacggcctggccgaagcacccgagcacggaaccacagccaccttccaccgctgcgccaaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggccttgaagttgccccatgtcgactacatcgaggaggactcctctgtctttgcccagagcatcccgtggaacctggagcggattacccctccacggtaccgggcggatgaataccagccccccgacggaggcagcctggtggaggtgtatctcctagacaccagcatacagagtgaccaccgggaaatcgagggcagggtcatggtcaccgacttcgagaatgtgcccgaggaggacgggacccgcttccacagacaggccagcaagtgtgacagtcatggcacccacctggcaggggtggtcagcggccgggatgccggcgtggccaagggtgccagcatgcgcagcctgcgcgtgctcaactgccaagggaagggcacggttagcggcaccctcataggcctggagtttattcggaaaagccagctggtccagcctgtggggccactggtggtgctgctgcccctggcgggtgggtacagccgcgtcctcaacgccgcctgccagcgcctggcgagggctggggtcgtgctggtcaccgctgccggcaacttccgggacgatgcctgcctctactccccagcctcagctcccgaggtcatcacagttggggccaccaatgcccaagaccagccggtgaccctggggactttggggaccaactttggccgctgtgtggacctctttgccccaggggaggacatcattggtgcctccagcgactgcagcacctgctttgtgtcacagagtgggacatcacaggctgctgcccacgtggctggcattgcagccatgatgctgtctgccgagccggagctcaccctggccgagttgaggcagagactgatccacttctctgccaaagatgtcatcaatgaggcctggttccctgaggaccagcgggtactgacccccaacctggtggccgccctgccccccagcacccatggggcaggttggcagctgttttgcaggactgtatggtcagcacactcggggcctacacggatggccacagccgtcgcccgctgcgccccagatgaggagctgctgagctgctccagtttctccaggagtgggaagcggcggggcgagcgcatggaggcccaagggggcaagctggtctgccgggcccacaacgcttttgggggtgagggtgtctacgccattgccaggtgctgcctgctaccccaggccaactgcagcgtccacacagctccaccagctgaggccagcatggggacccgtgtccactgccaccaacagggccacgtcctcacaggctgcagctcccactgggaggtggaggaccttggcacccacaagccgcctgtgctgaggccacgaggtcagcccaaccagtgcgtgggccacagggaggccagcatccacgcttcctgctgccatgccccaggtctggaatgcaaagtcaaggagcatggaatcccggcccctcaggagcaggtgaccgtggcctgcgaggagggctggaccctgactggctgcagtgccctccctgggacctcccacgtcctgggggcctacgccgtagacaacacgtgtgtagtcaggagccgggacgtcagcactacaggcagcaccagcgaaggggccgtgacagccgttgccatctgctgccggagccggcacctggcgcaggcctcccaggagctccagtgacagccccatcccaggatgggtgtctggggagggtcaagggctggggctgagctttaaaatggttccgacttgtccctctctcagccctccatggcctggcacgaggggatggggatgcttccgcctttccggggctgctggcctggcccttgagtggggcagcctccttgcctggaactcactcactctgggtgcctcctccccaggtggaggtgccaggaagctccctccctcactgtggggcatttcaccattcaaacaggtcgagctgtgctcgggtgctgccagctgctcccaatgtgccgatgtccgtgggcagaatgacttttattgagctcttgttccgtgccaggcattcaatcctcaggtctccaccaaggaggcaggattcttcccatggataggggagggggcggtaggggctgcagggacaaacatcgttggggggtgagtgtgaaaggtgctgatggccctcatctccagctaactgtggagaagcccctgggggctccctgattaatggaggcttagctttctggatggcatctagccagaggctggagacaggtgcgcccctggtggtcacaggctgtgccttggtttcctgagccacctttactctgctctatgccaggctgtgctagcaacacccaaaggtggcctgcggggagccatcacctaggactgactcggcagtgtgcagtggtgcatgcactgtctcagccaacccgctccactacccggcagggtacacattcgcacccctacttcacagaggaagaaacctggaaccagagggggcgtgcctgccaagctcacacagcaggaactgagccagaaacgcagattgggctggctctgaagccaagcctcttcttacttcacccggctgggctcctcatttttacgggtaacagtgaggctgggaaggggaacacagaccaggaagctcggtgagtgatggcagaacgatgcctgcaggcatggaactttttccgttatcacccaggcctgattcactggcctggcggagatgcttctaaggcatggtcgggggagagggccaacaactgtccctccttgagcaccagccccacccaagcaagcagacatttatcttttgggtctgtcctctctgttgcctttttacagccaacttttctagacctgttttgcttttgtaacttgaagatatttattctgggttttgtagcatttttattaatatggtgactttttaaaataaaaacaaacaaacgttgtcct

SEQ ID NO: 81 - DNA, 19 bp, artificial sequence
  synthetically generated oligonucleotide
aacgcaaggctagcaccag

SEQ ID NO: 82 - DNA, 19 bp, artificial sequence
  synthetically generated oligonucleotide
gcctccattaatcagggag

SEQ ID NO: 83 - DNA, 14 bp, artificial sequence
  synthetically generated oligonucleotide
gaccatgccttaga

SEQ ID NO: 84 - DNA, 16 bp, artificial sequence
  synthetically generated oligonucleotide
ggattgaatgcctggc

SEQ ID NO: 85 - DNA, 13 bp, artificial sequence
  synthetically generated oligonucleotide
agacagaggagtc

SEQ ID NO: 86 - DNA, 13 bp, artificial sequence
  synthetically generated oligonucleotide
agacaaaggagtc

SEQ ID NO: 87 - DNA, 13 bp, artificial sequence
  synthetically generated oligonucleotide
cccagagtgagtg

SEQ ID NO: 88 - DNA, 13 bp, artificial sequence
  synthetically generated oligonucleotide
cccagagtgaggg

SEQ ID NO: 89 - DNA, 13 bp, artificial sequence
  synthetically generated oligonucleotide
cggctgtacccac

SEQ ID NO: 90 - DNA, 13 bp, artificial sequence
  synthetically generated oligonucleotide
cggctatacccac

SEQ ID NO: 91 - DNA, 13 bp, artificial sequence
  synthetically generated oligonucleotide
ccttgactttgca

SEQ ID NO: 92 - DNA, 13 bp, artificial sequence
  synthetically generated oligonucleotide
ccttgattttgca

SEQ ID NO: 93 - DNA, 12 bp, artificial sequence
  synthetically generated oligonucleotide
atcgtcccggaa

SEQ ID NO: 94 - DNA, 12 bp, artificial sequence
  synthetically generated oligonucleotide
gtcgtcccggaa